(19) United States
(12) Patent Application Publication
(10) Pub. No.: US 2005/0244843 A1
(43) Pub. Date: Nov. 3, 2005

(54) Blood test prototypes and methods for the detection of circulating tumor and endothelial cells

(76) Inventors: Wen-Tien Chen, Stony Brook, NY (US); Lee Chen, Farmingville, NY (US); Che Chen, Stony Brook, NY (US)

Correspondence Address: PILLSBURY WINTHROP SHAW PITTMAN, LLP, P.O. BOX 10500, MCLEAN, VA, 22102, US

(21) Appl. No.: 10/978,029

(22) Filed: Oct. 30, 2004

Related U.S. Application Data
(63) Continuation-in-part of application No. 10/122,268, filed on Apr. 11, 2002, now abandoned.
(63) Continuation-in-part of application No. 10/220,347, filed on Aug. 28, 2002.
(60) Provisional application No. 60/516,571, filed on Oct. 31, 2003.
(60) Provisional application No. 60/332,408, filed on Nov. 16, 2001.

(51) Int. Cl. C12Q 1/68; G01N 1/30; G01N 33/48; C12M 1/34

(52) U.S. Cl. 435/6; 435/40.5; 435/287.1

(57) ABSTRACT

Methods and devices for isolating and diagnosing disease with a cell adhesion matrix system, mimicking a metastatic, cardiovascular or placental environment, are disclosed. The cell adhesion matrix facilitates the enrichment of target cells such as metastatic tumor cells, fetal cells and endothelial progenitor cells from a fluid sample such as blood for diagnostic and therapeutic applications in treating patients afflicted with disease, such as cancerous, cardiovascular and fetal diseases, as well as for research applications in molecular analysis of metastatic, and cardiovascular and fetal diseases. Blood test prototypes and methods for the cell enrichment and detection of circulating tumor and endothelial cells using multiplex molecular analysis are described herein. In addition, methods and compositions for determining host immunity to tumor in subjects with risk of cancer progression and methods for isolating an enriched fraction of fetal cells from pregnant females for prenatal diagnosis are also described herein.

### CROSS REFERENCE TO RELATED APPLICATIONS

This application claims benefit of priority from U.S. Provisional Patent Application Ser. No. 60/516,571, filed on Oct. 31, 2003 and is a continuation-in-part application of U.S. patent application Ser. No. 10/122,268, filed on Apr. 11, 2002, which claims benefit of priority from U.S. Provisional Patent Application Ser. No. 60/332,408, filed on Nov. 16, 2001, and is further a continuation-in-part of U.S. patent application Ser. No. 10/220,347 filed on Aug. 28, 2001 which claims benefit of priority from U.S. Provisional Application Ser. No. 60/231,517, filed on Sep. 9, 2000, all of which are herein incorporated by reference in their entirety.

### BACKGROUND OF THE INVENTION

1. Field of the Invention

The present invention generally relates to an improved cell adhesion matrix (“CAM”) and an improved cell isolation device for separating target cells such as tumor, fetal and angiogenic cells from blood or other tissue fluid samples such as ascites, scrape and smear specimens. More particularly, the present invention relates to a CAM system that may be used to selectively isolate cell, for example, target cancer cells with metastatic potential and/or endothelial progenitor cells that display invadopodia.

2. Description of the Related Art

Circulating Tumor Cells (CTC) And Cancer Detection

Malignant tumors of epithelial tissues are the most common form of cancer and are responsible for the majority of cancer-related deaths. Because of progress in the surgical treatment of these tumors, mortality is linked increasingly to early metastasis and recurrence, which is often occult at the time of primary diagnosis (Racila et al., 1998; Pantel et al., 1999). For example, the remote anatomical location of the pancreas and other gastrointestinal (GI) organs makes it unlikely that pancreatic and other GI cancers will be detected before they have invaded neighboring structures and grown to tumors larger than 1-cm (Compton, 2003; Flatmark et al., 2002; Koch et al., 2001; Liefers et al., 1998; Matsunami et al., 2003; Nomoto et al., 1998; Pantel et al., 1999; Walsh and Terdiman, 2003; Weihrauch, 2002). Even with respect to breast cancers, 12-37% of small tumors of breast cancer (<1 cm) detected by mammography already have metastasized at diagnosis (Chadha M et al., 1994; Wilhelm M C et al., 1991).

Evidence has accumulated in the literature showing that epithelial tumor cells found in the circulation represent the earliest sign of metastasis formation and that circulating tumor cells (“CTC”) can be considered an independent diagnostic for cancer progression of carcinomas (Beitsch and Clifford, 2000; Brandt et al., 2001; Feezor et al., 2002; Fehm et al., 2002; Ghossein et al., 1999; Glaves, 1983; Karczewski et al., 1994; Koch et al., 2001; Liefers et al., 1998; Luzzi et al., 1998; Matsunami et al., 2003; Molnar et al., 2001; Wang et al., 2000; Weitz et al., 1999; Wharton et al., 1999; Racila et al., 1998; Pantel et al., 1999). Given the same, reliable procedures to isolate cancer cells from the bloodstream would have significant impact in both clinical diagnostic and therapeutic applications of cancer (Racila et al., 1998; Pantel et al., 1999). A new tumor staging, called Stage Mi, has been proposed to indicate the presence of tumor cells in the circulation of patients with cancers. The staging warrants the development of a blood test that could detect circulating tumor cells (CTC). The cancer research field awaits novel tumor cell enrichment methods that can increase detection sensitivity, advantageously by at least one order of magnitude (Pantel et al., 1999), over existing methods.

Circulating Endothelial Progenitor Cells, Angiogenesis And Cardio-Vascular Risk

Endothelial-cell injury is an important stimulus for the development of atherosclerotic plaque (Ross, 1993). Circulating endothelial progenitor cells (“CEC”) that can be isolated from the mononuclear cell fraction of the peripheral blood, bone marrow, and cord blood, have been identified (Asahara et al., 1997; Hill et al., 2003) as indicative of endothelial-cell injury. Laboratory evidence suggests that these cells express a number of endothelial-specific cell-surface markers and exhibit numerous endothelial properties. It has been noted that when these cells are injected into animal models with ischemia, they are rapidly incorporated into sites of neovascularization.

In a pilot study, Hill et al., 2003 found that a low CEC level was associated with cardiovascular risk factors and with brachial reactivity. It has been suggested that endothelial injury in the absence of sufficient CEC might affect the progression of cardiovascular disease. This early-phase study pointed to the potential of CEC in diagnosis and treatment of cardiovascular diseases. CEC might contribute to endothelial repair by providing a circulating pool of cells to promote angiogenesis (Szmitko et al., 2003). Thus, CEC may be a negative predictor of the risk of cardiovascular diseases. An efficient enrichment method for CEC would be very useful therefore in pre-diagnosis of and management of cardiovascular disease.

Cell Heterogeneity and Current Cell Separation Technologies

Tumor and endothelial progenitor cells circulating in the blood (a heterogeneous source of cells) are rare. These cells can be hard to purify for analysis. In cancer patients, the number of CTC or exfoliated abnormal cells (neoplastic cells) in blood is generally very small compared to the number of non-neoplastic cells. Therefore, the detection of exfoliated abnormal cells by routine cytopathology is often limited. Further, exfoliated cells are frequently highly heterogeneous being composed of many different cell types (interestingly, many of the genes initially reported to be differentially expressed in exfoliated cells have actually turned out to be expressed by non-tumor cells instead). Compounding this heterogeneicity problem, the frequency of neoplastic cells present in each clinical specimen is variable, which biases and complicates the quantification of differential gene expression in randomized mixed population. Apoptotic and necrotic cells are common in larger tumors, peripheral blood and ascites. These cells do not contain high quality RNA and thus present technical problems for molecular analyses (Karczewski et al., 1994).

A number of cell enrichment methods for circulating tumor and endothelial progenitor cells have been described:

a) Microdissection can be used to isolate rare tumor cells one by one (Suarez-Quian et al., 1999). This method typically has several limitations: (1) the subsequent sample processing is complicated, (2) cell viability cannot readily be established, and (3) selection of the cells to be dissected is based mainly on morphological criteria, which has a high frequency of giving rise to false-positive results.

b) Physical characteristics of tumor cells, such as shape, size, density or electrical charge, can also be used (Vona et al., 2000). Several density gradient centrifugation methods have been developed to enrich tumor cells in nucleated blood cells (devoid of mature red blood cells). Density gradient centrifugation methods can achieve 500 to 1,000-fold cell enrichment. The enriched tumor cells can then be subjected to molecular analysis using highly sensitive assays such as immunocytochemistry and reverse transcriptase polymerase chain reaction (RT-PCR) which may be used to amplify putative tumor markers or epithelial markers such as prostate specific antigen (PSA) mRNA or cytokeratin 19 mRNA (Peck et al., 1998). However, these methods may not effectively enrich viable tumor cells from normal cells. That is, 500-1,000 fold cell enrichment is often found to be relatively modest enrichment which generates substantial background noise adversely affecting further molecular analysis. In addition, enrichment methods based on physical separation techniques are often cumbersome, lengthy, and involve steps (e.g. more than 2-3 rounds of centrifugation) that can result in cellular damage.

c) Antibody-based techniques are a more recent development. Immunoaffinity methods include affixing an antibody to a physical carrier or fluorescent label. Sorting steps can then be used to positively or negatively enrich for the desired cell type after the antibody binds to its target present on the surface of the cells of interest. Such methods include affinity chromatography, particle magnetic separation, centrifugation, or filtration, and flow cytometry (including fluorescence activated cell sorting; FACS).
(1) Flow cytometry or a fluorescence activated cell sorter (“FACS”) detects and separates individual cells one-by-one from background cells. In model experiments, this method can detect breast carcinoma cells (Gross et al., 1995) and endothelial progenitor cells (Hill et al., 2003) in the mononuclear cell fraction that had been enriched from the peripheral blood by density gradient centrifugation. Furthermore, FACS can detect naturally occurring breast and prostate tumor cells in blood after an enrichment step using antibody-coated magnetic microbeads (Racila et al., 1998; Beitsch and Clifford, 2000). However, cells that exist in clusters or clumps are discarded during the FACS process, and in some instances, for example, ovarian cancer, most of the cells are present as aggregates, making FACS CTC or CEC detection highly ineffective.
(2) Approaches based on antibody-coated microbeads can use magnetic fields (Racila et at, 1998), column chromatography, centrifugation, filtration or FACS to achieve separation. Despite its great power for enrichment, there are also inherent limitations associated with all of the antibody-based cell separation methods. The most serious one is that cancer cells usually express putative tumor-specific antigens to variable degrees (Sabile et al., 1999); hence it is easy to lose a large and potentially non-random subset of tumor cells during the collection. Antibodies also tend to bind with significant non-specific affinity to damaged cells, leading to their co-purification with the cells of interest. Overall, such antibody-based cell separation methods have a higher than desired false-negative rate. Current antibody-initiated magnetic separation methods have detected CTC at much lower levels, i.e., 1-100 CTC per mL of blood from patients with breast and prostate cancer (Racila et al., 1998), or less than 50 CEC per mL of blood of individuals at risk of cardiovascular diseases (Hill et al., 2003; Beitsch and Clifford, 2000). There are approximately 5×10⁹ red cells and 5×10⁶ white nucleate cells present in one milliliter (mL) or gram of blood. Therefore, it is still a challenging task to detect the presence of thousands of cancer or endothelial cells in one mL of blood (Gulati and Acaba, 1993).

Over the past 20 years, specialized complexes found on the surface of invasive tumor cells that facilitate their movement from the primary tumor to sites of metastasis have been characterized (Aoyama and Chen, 1990; Chen and Chen, 1987; Chen et al., 1994a; Chen et al., 1984; Chen et al., 1994b; Chen, 1996; Chen, 1989; Chen and Wang, 1999; Ghersi et al., 2002; Goldstein and Chen, 2000; Goldstein et al., 1997; Kelly et al., 1994; Monsky et at, 1994; Monsky et al., 1993; Mueller et al., 1999; Mueller and Chen, 1991; Mueller et al., 1992; Nakahara et al., 1996; Nakaliara et al., 1998; Nakahara et al., 1997; Pavlaki et al., 2002; Pineiro-Sanchez et al., 1997; Saga et at, 1988; Zucker et at, 2000; Zukowska-Grojec et al., 1998). These complexes, which we have denoted as “invadopodia”, bind to and degrade multiple types of endothelial cell matrix (ECM) components. Invadopodia are not found on differentiated normal blood cells or on primary tumor cells, and they do not function effectively on dead or dying cells. Invadopodia are present in circulating endothelial progenitor cells but not in more than 99.999% of blood cells, and in fetal cells found in maternal blood of pregnant females. The present inventors have recognized an enrichment step based on invadopodia function would powerfully serve to separate viable metastatic tumor cells and endothelial cells from the majority of cell types found in ascites, blood, and many other body fluids and would address the limitations of the other technologies described above.

### SUMMARY OF THE INVENTION

In one embodiment, there is provided CAM for isolating specific viable target cells in a blood sample or other tissue fluid sample for use in the screening, diagnostic evaluation, prognosis and management of disease.

A CAM of the present invention utilizes a cell-adhesion material about a core material to effectively promote the adhesion of target cells including, CTC and CEC. Useful cell-adhesion materials include blood-borne adhesion compounds and include, without limitation, fibronectin, fibrin, heparin, laminin, tenascin or vitronectin, and synthetic compounds, such as synthetic fibronectin and laminin peptides, extra cellular matrix compounds, or fragments thereof, combinations thereof, and the like. Useful cell-adhesion materials in a CAM should have the ability to effectively coat the core material of the matrix alone, or in combination with other materials. The core preferably comprises a chemically non-reactive material such as, but not limited to, gelatin particles, bone fragments, collagen, glass beads, inert polymeric materials (such as magnetic colloid, polystyrene, polyamide materials like nylon, polyester materials, cellulose ethers and esters like cellulose acetate), urethane DEAE-dextran, as well as other natural and synthetic materials, such as foam particles, cotton, wool, dacron, rayon, acrylates and the like. The CAM may be applied to form a coat, such as from about 1.0-1.5 mm in thickness.

For example, a CAM might comprise gelatin particle or glass bead core materials coated with a type I collagen solution that is then polymerized to form a film. The film containing such porous collagen-coated beads can then be exposed to a sample, such as serum or whole blood containing one or more blood-borne adhesion components that promote the adhesion of a target cell, such as CTC and CEC. Blood-borne adhesion materials that promote adhesion of cells such as CTC and CEC may comprise, for example, basement membrane components such as fibronectin, fibrin, laminin, heparin, and vitronectin, fragments thereof, combinations thereof, or biological mimics of these components, and modified versions thereof as seen in extravasation or endothelial injury, and may be prepared by purification from natural sources or synthesized by artificial means. A CAM may further comprise specific ligands which also recognize and bind target cells with a high degree of sensitivity and specificity.

The CAM film may include microbeads, such as type I collagen coated gelatin-microbeads or glass-microbeads, covered with blood borne-cell adhesion molecules, such as those present in blood or body fluids, and a binding material. For example, microbeads may comprise (but are not limited to) dehydrated gelatin particle or glass beads, with diameter in the range of 200 microns to 2,000 microns. In one embodiment, the microbeads are configured, or of such shape and size, to create anastomosic channels allowing blood flow in the film.

In embodiments wherein the target cells are CTC and CEC, the CAM film of the invention preferably has an affinity and specificity for the target cells, CTC and CEC, with minimal affinity for other cells, such as a small fraction of hematopoietic cells. The CAM film may be designed to mimic the site at the vessel wall of arteriovenous anastomosis or loci of metastases or cardiovascular plaques, where extracellular matrix (ECM) components, including collagens, proteoglycans, fibronectin, laminin, fibrin, heparin, tenascin and vitronectin etc., have been modified during the process of extravasation or endothelial injury. In essence, the CAM composition and assay surface architecture may be designed, using the information presented herein, to improve mimicry of the cell microenvironment so as to enable a more maximal number of viable target cells, such as CTC and CEC, to be recovered from whole blood. The target cells, including CTC and CEC, isolated by the methods of this invention are typically viable, may exhibit growth ex vivo, and may exhibit the adhesive activity against extracellular matrix components, ECM. Isolated CTC and CEC from blood may be used to establish an expression profile of CTC and CEC.

A CAM of the present disclosure may be used, for example, in the detection, diagnosis and management of cancer. The CAM may be used to recognize and bind with high affinity and specificity to viable cancer cells, and therefore, the matrix may be used to isolate cancer cells from fluid samples such as blood samples and/or ascites fluid taken from a patient suffering with cancer. The CAM may be used for capturing metastatic cancer cells in the patient's sample for the diagnosis and monitoring of the disease in such patients inflicted with cancer. CAMs may be used to detect and isolate viable circulating metastatic tumor cells from all types of cancers, including, ovarian, lung cancer such as non-small cell and small cell lung cancer, prostatic, pancreatic, breast cancer, melanoma, liver, stomach, cervical, renal, adrenal, thyroid, and adenocarcinomas such as colorectal cancer.

Alternatively, the matrix can be used to capture endothelial cells in blood samples for the detection, diagnosis and management of cardiovascular disease in a patient. CAM has the ability to bind with high affinity and selectivity to viable endothelial cells present in the blood sample when a blood sample taken from a patient having cardiovascular disease is contacted with the matrix. Endothelial cells at various stages of development, including progenitor endothelial cells, may be used in diagnosis of cardiovascular disease, such as angiogenesis in patients inflicted with this disease.

The present invention also provides a cell isolation device utilizing the CAM of the present invention to isolate target cells from fluid samples such as blood. Such device may provide, for example, an “endothelial cell trap” that allows for the efficient enrichment and identification of target cells, wherein the target cells are, for example, viable endothelial progenitor cells in the peripheral blood of a subject with risk of cancer and/or cardiovascular diseases. A CAM-initiated cell isolation device may be designed to provide a one million-fold enrichment of viable circulating tumor cells and circulating endothelial cells from blood.

In another embodiment, the CAM can be used to capture and isolate target cells such as fetal cells present in the maternal circulation of pregnant females. The isolated cells adhering to the CAM can then be used for analysis in prenatal diagnosis of diseases such as Down's Syndrome, Marfan's Syndrome, Taysach's disease and others using standard procedures. Isolating fetal cells using the present matrix allows for a safer method for prenatal diagnosis of disease, since the fetal cells can be isolated directly from a blood sample and no invasive procedures of the pregnant mother are necessary. In this and other embodiments of the invention, the CAM enriches or increases the number of cells that would normally be available for analysis in a blood sample using standard techniques of cell isolation.

Using the present disclosure, CAM cell enrichment may be designed to have one or more of the following features: (a) a one-million-fold enrichment of viable target cells, including CTC and CEC, from whole blood with a high degree of sensitivity and specificity for the target cells necessary for the diagnosis of disease; (b) concurrent functional and morphological discrimination, for example, cell size and density, of the target cells, including CTC and CEC, from other normal blood and tissue cells; (c) whole blood may be used as the starting sample or cell fractions prepared by a common density gradient centrifugation procedure. CAM cell enrichment may be a single or multistep process.

Further disclosed is a CAM-initiated cell isolation device that permits efficient captures of viable target cells, including CTC and CEC, from the mononuclear cell population. Target cells may be fractioned from blood or tissue fluid samples derived from subjects inflicted with a disease such as cardiovascular disease or cancer, as discussed in co-pending application PCT Patent Application PCT/USO1/26735—claiming priority to U.S. Provisional Patent Application No. 60/231,517 (the disclosure of which is incorporated herein by reference in its entirety). Such a device may comprise, for example, a CAM coating that is preferably immobilized to the surface of a vessel, such as, but not limited to, the inner bottom surface of a tube, a surface of a slide, or the inner bottom surface of a Petrie dish. The matrix-coated surfaces of the CAM-initiated cell isolation vessels are preferably designed to maximize contact for the sample when sample is placed into the vessel. The CAM-initiated cell isolation device may make use of a variety of already available laboratory diagnostic vessels, for example, a cell culture chamber slide, a culture microtiter plate, a culture flask, etc.

The CAM-initiated cell isolation device may be rotated to more optimally imitate blood flow to increase contact between the cells and CAM, thus promoting more efficient enrichment (of, for example, viable CTC and CEC).

A CAM-initiated blood device may be constructed based on the present disclosure that is more efficient in removing viable target cells including, CTC from the peripheral blood of a subject suffering with, for example, CTC related disease, than that described in co-pending application PCT Patent Application PCT/USO1/26735 (claiming priority to U.S. Provisional Patent Application No. 60/231,517).

The methods and CAM films described above for enrichment of tumor cells may also readily be used as a negative filtration step for harvested autologous blood or bone marrow to remove cancer cells. A CAM-initiated blood filtration device of the present disclosure may be employed to remove contaminating cancer cells, for example, in respect of the auto transfusion of blood salvaged during cancer surgery, therapeutic bone marrow transplantation, peripheral blood stem cell transplantation and aphaeresis, in which autologous transfusions are done, Further, the described CAM-initiated blood filtration unit may be used to prevent full blown cancer from occurring by removing cells capable of metastasis from the circulation.

CAM-initiated blood filtration may similarly be utilized in the preparation of cancer-free autologous bone marrow cells intended for replacement after aggressive, bone-marrow chemotherapy - radiation in cancer patients. Detection of cancerous cells may be improved by molecular amplification techniques, and CAM-enriched cells may be used in multiplex molecular analysis such as tests for DNA, proteins and immunological tests (as, for example, specific for CTC and CEC from a subject).

CAM-enriched cells and their DNAs, RNAs, proteins or antigens may be applied to multiplex detection assays for cancer diagnostic purposes. Cell markers used in the multiplex CTC detection assay include, but not limited to, the CTC invasive phenotype [collagen ingestion and acetyl LDL uptake by the cell], the epithelial antigens [cytokeratins, epithelial specific antigens (EPCAM, HEA, Muc-1, EMA, GA733-1, GA733-2, E-cadherin, EGFR, TAGI2, lipocalin 2 (oncogene 24p3)], endothelial antigens [CD31/PECAM1, van Willebrand factor (vWF), FIt-1 (a receptor for VEGF), VE-cadherin] and other tumor associated antigens [including, but not limited to, carcinoembryonic antigen (CEA), epidermal growth factor receptor (EGFR), human kallikrein-2 (HK2), mucin (MUC), prostate-specific antigen (PSA), prostate-specific membrane antigen (PMA), 13 subunit of human chorionic gonadotropin (13-hCG) etc.]. Markers may be applied individually or jointly to achieve the effective identification and enumeration of viable tumor cells in a given volume of the blood or body fluids from a subject. The methods for data readouts include, but are not limited to, flow cytometry, fluorescent microscopy, enzyme-linked immunoabsorb assay (ELISA), and quantitative real-time RT-PCR etc.

CAM-enriched CTC cells provide sources for genetic testing for cancer. The alterations in gene structure and function that may be genetically tested in CTC cells include, but are not limited to, oncogenes (e.g., ERBB2, RAS, MYC, BCL2, etc.), tumor suppression genes (e.g., p53, APC, BR CA], BRCA2, CDKN2A, CCND1, CDC2SA, CDC25B, KIP], RB] etc), genes associated with tumor progression [e.g., carcino-embryonic antigen (CEA), epidermal growth factor receptor (EGFR), human kallikrein-2 (HK2), mucin (MUG), prostate-specific antigen (PSA), prostate-specific membrane antigen (PMA), 13 subunit of human chorionic gonadotropin (13-hCG), etc.], and genes associated with metastatic cascades [e.g., nm23 family (HJ-6) of necleoside diphosphate kinases (cell migration), PTEN/MMAC] (cell migration and focal adhesions), CADJ/E-cadherin (cell-cell adhesion), MKK4/SEKi (cellular response to stress), KISS-i (regulation of MIIMLP9 expression), BR/VISI (cell motility) etc]. For example, aneuploidy and CKi9, ERB2, CEA, MUG], EGF receptor, J3-hCG alterations are useful in diagnosis of breast cancer; pS3, Ki-ras mutations CDKN2A, LOH 3p, FHIP for lung cancer; p53, APC, CEA, CKi9, CK20, ERBB2, Ki-ras mutations for colorectal, gastric, and pancreatic cancers; PSA, PSM, HK2 for prostate cancer; p53 mutations and microsatellite alterations for head and neck cancer. The genetic markers may be applied individually or jointly to achieve the effective detection of genetic changes in a subject. The methods for data readouts include, but limited to, flow cytometry, fluorescent microscopy, fluorescent or color based polymerase chain reaction readers etc.

CAM-enriched CEC cells and their DNAs, RNAs, proteins or antigens currently known in a specific tumor may also be applied to multiplex CEC detection assays for detecting subjects with risk of cardiovascular diseases. The cell markers used in the multiplex CEC detection assay include, but are not limited to, the CEC functional phenotype [acetyl LDL uptake by the cell] and endothelial antigens [CD3 1/PECAM-1, van Willebrand factor (vWF), Flk-1 (a receptor for VEGF), VE-cadherin]. The markers may be applied individually or jointly to achieve the effective identification and enumeration of viable endothelial cells in a given volume of blood or body fluids from a subject. Methods for data readouts include, but are limited to, flow cytometry, fluorescent microscopy, enzyme-linked immunoabsorb assay (ELISA), and quantitative real-time RT-PCR, etc. CAM-enriched CEC cells may further provide a source for genetic testing of a subject. That is, alterations in gene structure and function of a subject may be genetically tested using the CTC cells enriched by CAM. The genetic markers may be applied individually or jointly to achieve the effective detection of genetic changes in a subject.

In one embodiment, viable cells captured on the CAM can be released readily from the device surface by the use of digestive enzymes, including, but not limited to, collagenases, trypsin/EDTA solution (purchased from GIBCO), and hyaluronases by selecting appropriate core materials and cell adhesion coatings. For example, cell adhesion molecules and collagen or gelatin of the CAM film may be sensitive to digestion. Enzymes that will cleave binding between the cells and the matrix, will release viable cells from the CAM film into suspension. For example, CAM-captured cells may be effectively released into suspension using collagenase when type I collagen is the skeleton supporting the cell adhesion molecules.

The detection methods of the present invention may be used for cancer diagnostic purposes, e.g. early detection, monitoring therapeutic and surgical responses, and prognostication of cancer progression. CAM-enriched CTC may be used, for example, to detect cancer earlier than using current surgical methods of isolating tumor cells, to monitor therapeutic and surgical responses, to improve the accuracy of cancer staging, and to determine the metastatic potential of the patient's tumor. These applications may be further enhanced using additional multiplex molecular assays known to those of skill in the art, such as determining the genetic alterations of a subject, verifying the tissue origin of circulating tumor cells, measuring the molecular markers of the types of cancer, and determining the degree of reduction in tumor cytotoxic leukocyte count or complement association.

Prognosis and therapeutic effectiveness may also be adjudged by the detection assays of the present invention. For example, the count of viable CTC during and post therapeutic intervention(s) may be used to ascertain therapeutic effectiveness. CAM-enriched CTC and associated anti-tumor host immunity may be detected and quantified in conjunction with microscopic imaging and flow cytometry. Selection of chemotherapeutic regimen may be optimized by determining those regimens that most effectively, without undue side effects, reduce the number of viable CTC in the blood sample. Optimization of selection of chemotherapeutic regimen may also be performed by subjecting the CAM-enriched CTC to a battery of chemotherapeutic regimes ex vivo. Effective doses or drug combinations could then be administered to that same patient. The number of viable CTC can be determined before and after the administration of the compound or agent. Compounds or agents that significantly reduce the number of viable CTC after administration may be selected as promising anti-cancer agents. Agents exhibiting efficacy are those, which are capable of decreasing number of CTC, increasing cytotoxic leucocytes and complement system (host immunity), and suppressing tumor cell proliferation.

The detection methods of the present invention may also be used to detect whether a new compound or agent has anti-cardiovascular disease, or other activity.

It should be noted that most CTC are dead or apoptotic in the circulation due to the presence of host immunity to tumors, as described in co-pending PCT Patent Application PCT/US01/26735. The viability of CTC and tumor associated cytotoxic leukocytes, and measurements with respect to the autologous complement system derived from individual donors put together an effective means of determining host immunity against tumors. A subject may be considered as having anti-tumor immunity, when the number of viable CTC enriched by CAM is high in the absence of autologous plasma but low in the presence of autologous plasma. On the other hand, a subject who loses anti-tumor immunity would have high levels of viable CTC in the presence and absence of autologous plasma that resist immune killing.

Viable CTC enriched from blood of cancer patients by a CAM method may also be used in fusions with dendritic cells for anti-cancer vaccine development. For example, the CTC from individual patients with different cancers may be subjected to ex vivo culture and expansion, and the cells may be used in whole, or purified for specific membrane structures or for specific antigens, to interact with dendritic cells for the development of an effective tumor vaccine.

Cytotoxic lymphocytes enriched by the CAM methods from blood of cancer patients may be valuable in their own right: careful comparison of their gene expression profile in comparison to non-tumor associated lymphocytes may yield valuable information concerning the type of ongoing immune reaction and inflammation that are being mounted against the metastatic tumor cells. Moreover, another valuable therapy approach may be to expand these cells in vitro, for example, using IL-2, and then reintroduce them into the patients to augment their anti-tumor immune response. This approach may have dramatic utility in the management of melanoma and other tumors.

Embodiments of the present invention would be useful both for diagnostic and therapeutic purposes in providing the ability to separate, for example, the small fraction of CTC that are metastatic from the large number of other circulating cells in a patient's body.

Embodiments of the present invention: (1) can isolate specifically viable target cells such as tumor and endothelial cells but leave alone unrelated or damaged cells; (2) can achieve an enrichment of over one hundred target cells such as tumor or endothelial cells, from over five billion cells in whole blood; (3) can identify target cells such as “cancer cells” or “endothelial progenitor cells” from normal blood cells readily in the same assay format; (4) can enrich cells from background normal blood cells that are useful in diagnosis and treatment of patients suffering with a disease such as metastatic cancers and cardiovascular diseases.

### BRIEF DESCRIPTION OF THE DRAWINGS

FIG. 1A depicts a front sectional view of a CAM 16-well chamber slide whose bottom surface is coated with a CAM film, such as a fluorescently labeled collagen film, capable of enriching circulating tumor cells and endothelial progenitor cells that may be used in the diagnosis of cancer and cardiovascular diseases;

FIG. 1B depicts a front sectional view of a CAM 96-well chamber slide whose bottom surface is coated with a CAM film, such as a fluorescently-labeled CAM film, comprising collagen that is capable of enriching circulating tumor cells and endothelial progenitor cells and that may be used in the diagnosis of cancer and cardiovascular diseases;

FIGS. 2A, 2B and 2C depict a front sectional view of upright 7 ml, 15 ml and 30 ml vacuum blood collection tubes that may be used in the diagnosis of diseases that are coated along their internal surface with a CAM film;

FIG. 2D depicts a front sectional view of an upright tissue culture bottle coated along its internal surface with a CAM film that may be used in the diagnosis and treatment of cancer and cardiovascular diseases;

FIG. 2E depicts an enlarged front sectional view of a CAM film in a vessel such as in FIGS. 2A-2D;

FIG. 3A depicts a front sectional view of an upright blood collection tube with a dipstick insert coated with a CAM film;

FIG. 3B depicts a front sectional view of the dipstick of FIG. 3A;

FIG. 4A depicts a three-dimensional view of a blood filtration cassette containing a pre-filter mesh inlet in the housing for the introduction of the sample to be filtered; a main filter compartment filled with cell separation beads coated with a thin CAM film; a post-filter mesh outlet in the housing for the removal of filtered blood, which may be used in conjunction with a blood filtration system for diagnostics, therapeutics or treatment according to the invention; and

FIG. 4B is an expanded cross-sectional view of the main filter compartment of FIG. 4A filled with cell separation beads coated with a CAM film depicting the anastomosic channels formed by the cell separation beads within the inner confinement area.

FIG. 5 is a immunocytochemistry micrograph of leukocytes (A) and tumor cells (B)/(C)/(D) derived from ascites of adenocarcinoma of the ovary enriched by a cell adhesion matrix using antibodies directed against CD45, a pan-leukocyte antigen, and pan-cytokeratins (B)/(C) or CD-31 (D) without (A)/(B) and without (C)/(D) antibody EpCA of positive-selection.

FIG. 6A-C is a real-time RT-PCR relative expression analysis of the expression of 10 genes selected from DNA microarray clusters with respect to tumor cells from ascites (FIGS. 6A and 6B) and tumor cells from a solid primary tumor (FIGS. 6A and 6C).

### DETAILED DESCRIPTION OF EMBODIMENTS OF THE INVENTION

The invention is directed to the isolation and detection of target cells in fluid samples taken from a patient for screening, diagnosis and management of diseases such as cancer and cardiovascular disease, and in prenatal diagnosis.

The isolation of target cells from fluid samples taken from a patient is facilitated by the present methods. Isolation of such cells may be useful in managing a disease state associated with such cells. For example, tumor and endothelial cell identification in blood samples taken from a patient are indicative of metastatic cancer and cardiovascular disease, respectively. Similarly, fetal cells present in a pregnant female's blood, therefore, can be isolated and used in prenatal diagnosis of disease associated with the fetus.

Embodiments of the invention involve target cell separation including tumor, endothelial, and fetal cells separation strategy using a functional enrichment procedure that captures the target cells based on an adhesive phenotypic behavior of invadopodia. This cell adhesion properties, which manifests as the propensity to bind with tight affinity and specificity to ECM matrices that mimic the blood vessel microenvironment, appears to be mediated by not one specific protein, but rather by a complex of proteins including specific cell adhesion receptor integrins that cluster on the cell surface in projections of cells denoted as “invadopodia.”

CAM Cell Enrichment

The tumor and endothelial cell separation strategy of CAM cell enrichment involves using a functional enrichment procedure that captures the target cells based on an adhesive phenotypic behavior to materials, as characterized in detail over the past decades (Aoyama and Chen, 1990; Chen and Chen, 1987; Chen et al., 1994a; Chen et al., 1984; Chen et al., 1994b; Chen, 1996; Chen, 1989; Chen and Wang, 1999; Ghersi et al., 2002; Goldstein and Chen, 2000; Goldstein et al., 1997; Kelly et al., 1994; Monsky et al., 1994; Monsky et al., 1993; Mueller et al., 1999; Mueller and Chen, 1991; Mueller et al., 1992; Nakahara et al., 1996; Nakahara et al., 1998; Nakahara et al., 1997; Pavlaki et al., 2002; Pineiro-Sanchez et al., 1997; Saga et al., 1988; Zucker et al., 2000; Zukowska-Grojec et al., 1998). It has been found that cells having invadopodia (“invadopodic cells”) bind with tight affinity to matrices that mimic the blood vessel microenvironment, especially in the perturbed state. Based on invadopodia behavior, a functional cell enrichment step that is highly selective for viable metastatic tumor cells and angiogenic endothelial cells and which captures few of leukocytes/monocytes and red cells, and leaves in solution other cell types may be designed. The CAM cell enrichment assay may additionally include a negative identification/selection procedure using antibodies directed against the leukocyte common antigen CD45.

The present method employs a CAM comprising biochemically a non-reactive core, such as collagen polymer, physically-associated with cell adhesion molecules, in particular natural and synthetic blood-borne adhesion molecules. A CAM preferentially is designed to permit viable tumor cells to adhere to the matrix while avoiding adherence to normal background cells in the blood; that is, allowing viable tumor cells to attach with great avidity but avoiding attachment to normal cells (preferably including, for example, more than 99.9% of white cells and 99.9999% of red cells) and dead or dying tumor cells. The CAM coating may also comprise a ligand (e.g., antibodies, fluorescent and/or colorimetric markers, etc.) capable of reacting with one or more CAM-invading cells. The ligand may cause a visible or non-visible (but detectable) change in the CAM indicative of the presence of one or more cells to be detected. Such ligands may alternatively in tandem be placed in a separate detection layer associated with the CAM. A thin CAM coating is preferably immobilized to the inner bottom surface of the cell separation unit.

Thus, CAM can be used to successfully recover viable tumor cells from, for example, the mononucleate cell fraction of blood samples from patients with stage I and IV non-small-cell lung cancer (NSCLC).

The CAM approach can also be used to mark tumor cells for the purpose of identification. For example, when the CAM is prepared using fluorescently labeled collagen, the invasive tumor cells become labeled, since they exhibit a propensity to digest and ingest collagen. In contrast, normal cells leave the CAM undisturbed.

In respect of invadopodic cells desired to be enriched, the CAM composition and assay surface architecture may be designed to improve mimicry of the intravascular microenvironment so that the maximal numbers of viable desired cells are recovered from a sample, such as whole blood. More efficient enrichment of the invadopodic cells may also be accomplished by use of a unit rotation procedure to optimally imitate blood flow and increase contact between the tumor cells and CAM. In a preferred embodiment, the sample typically should be processed in a manner to provide for retention of the viability of the invadopodic cell in the sample.

Cam-Initiated Cell Isolation Device

A CAM-initiated cell isolation device may comprise numerous designs such as a cell culture chamber slide, a culture microtiter plate, or a culture flask, etc.

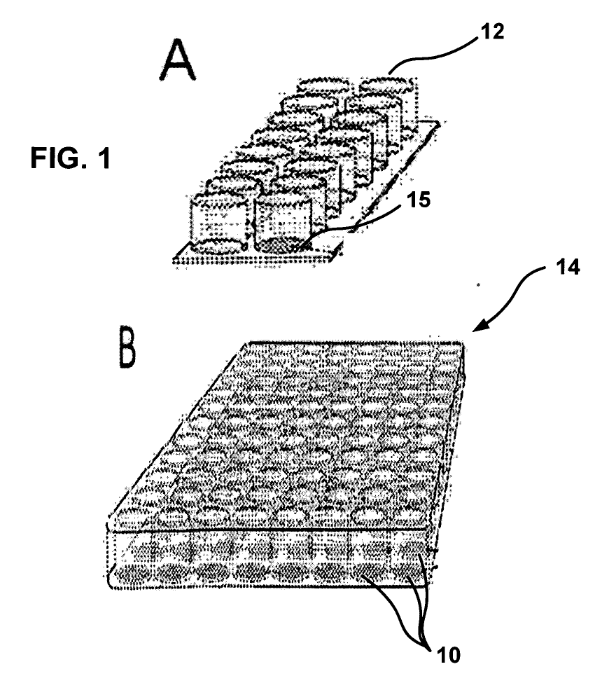

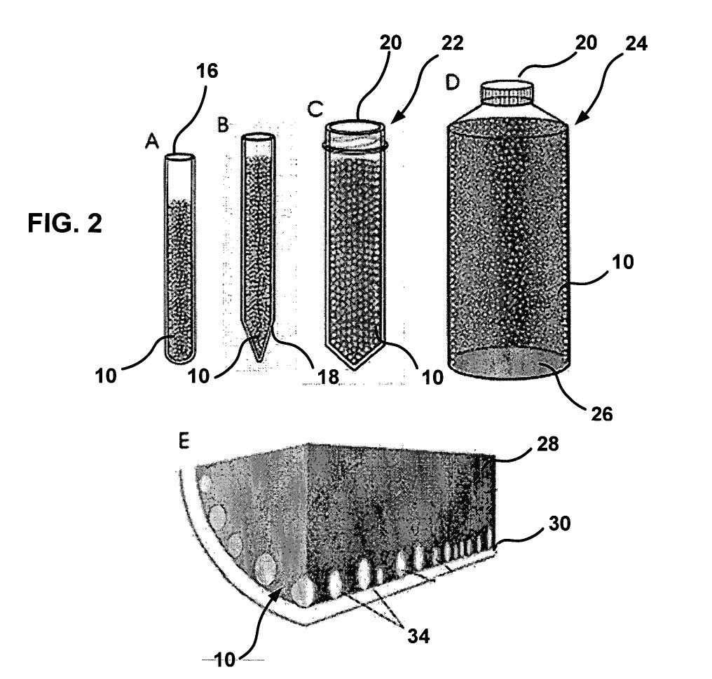

For example, the CAM-initiated cell isolation device may, as shown in FIG. 1, comprise a plurality of wells (12) in a unit array (14) having a CAM (10) at the bottom of one or more wells (12). FIG. 1A illustrates a 13-well microarray while FIG. 1B illustrates a 96-well microarray. The CAM-initiated cell isolation device may comprise a blood collection tube of various shapes (16, 18, 22) which may or may not be fitted with a cap (20) or a container (24) such as shown in FIG. 2, where the inner walls are coated with a CAM film (10), the bottom surface (26) uncoated, and fitted or not fitted with a cap (20). Preferably such vessels are sterilized before use. The CAM-initiated blood device may be used, for example, to isolate CTC and/or CEC in the CAM (30) from samples (28) placed in the vessel. The CAM (10) may be comprised, for example, of glass beads (34) incorporated within a layer (30) comprising a cell adhesion material.

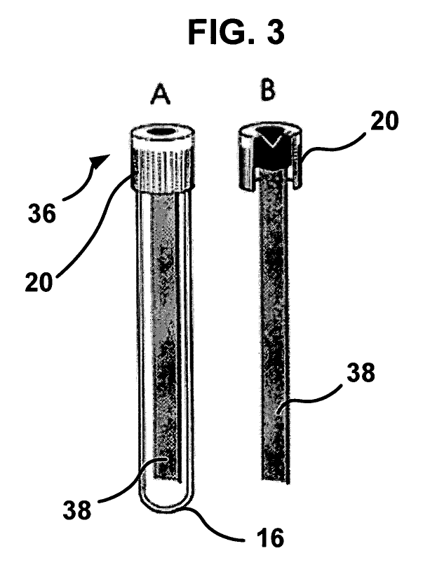

The CAM-initiated cell isolation device may utilize a dipstick (36) comprising a measuring card (38) such in perspective view and sectional view in FIG. 3B, the surface of the measuring card (38) being coated with CAM film. The dipstick or measuring card is inserted in a cell separation vessel (16). The CAM film may be spread over the surface of a dipstick (38) and/or the inner wall of the tube (16) and/or cap (20).

In one embodiment, the CAM-initiated cell isolation device further includes pre- and/or post-separation features such as filters (e.g., Amicon filters, hollow filters), membranes, or gradients (such as ficoll, sucrose, etc.) that help separate out cell populations before the population contacts the CAM film.

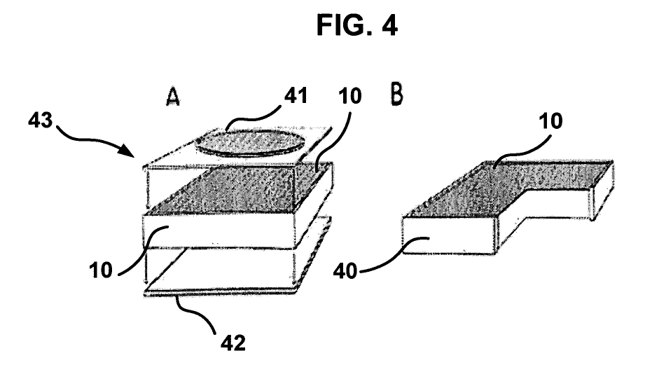

Turning to FIG. 4, there is shown a three-dimensional view of a blood filtration cassette (43) containing a pre-filter (41) such as a mesh (or CAM-coated mesh) in the housing for the introduction of the sample to be filtered, a main-filter compartment (40) filled with a CAM (10) and a post-filter (42) outlet in the housing. FIG. 4B is an expanded cross-sectional view of the main-filter compartment (40) filled with CAM (10).

In one embodiment, the CAM film of the CAM-initiated cell isolation device comprises collagen-coated microbeads, advantageously with a diameter in the range of 200 microns to 2,000, microns configured to create anastomosic channels allowing blood flow in the film. Whole blood in this blood filtration unit may be incubated at about 37° C. and rotated to imitate blood flow that increases contact between cells and CAM and supports efficient enrichment of viable cells from blood. Blood containing target cells such as tumor and endothelial progenitor cells may be stored in a CAM-initiated enrichment device for extended periods of time ranging from 4 to 48 hours to add efficiency of enrichment.

Three parameters may need to be addressed in designing a CAM-initiated cell isolation device and system: (i) the CAM composition and assay surface architecture to improve mimicry of the tumor intravascular microenvironment so that maximal numbers of viable tumor cells are recovered from whole blood; (ii) the unit rotation procedure to optimally imitate blood flow, increase contact between the tumor cells and CAM, and promote more efficient enrichment of viable tumor cells; and (iii) the blood process mode to improve retention of tumor cell viability in the blood samples.

The positive CTC selection method described above to enrich tumor cells may also be used as a negative filtration step for harvested autologous blood or bone marrow to remove cancer cells. The CAM-initiated blood filtration method of the invention thus may be employed in respect of the autotransfusion of blood salvaged during cancer surgery, therapeutic bone marrow transplantation, and peripheral blood stem cell transplantation and aphaeresis. The described CAM-initiated blood filtration unit may also be used to prevent full blown cancer from occurring by removing cells capable of metastasis from the circulation.

Specificity and sensitivity control experiments may be performed to optimize an assay's tumor cell enrichment efficiency. Significant variables include: (a) the viability of the exogenously added tumor cell lines after capture by CAM, (b) the conditions that most effectively enrich and isolate viable tumor cells, and (c) the cell processing mode that leads to complete elution of the cells from the CAM film.

### EXAMPLE 1

### CTC and CEC from Blood

Whole blood may be placed in a CAM blood collection unit, such as a blood collection tube (FIGS. 2 and 3). The tube may be incubated at about 37° C. and rotated to imitate blood flow so as to increase contact between cells and CAM. Blood may be collected in the presence of anticoagulants, i.e., Anticoagulant Citrate Dextrose solution USP (ACD, Baxter Healthcare Corporation, Deerfield, Ill.) plus 50 units of lithium heparin per mE, to prevent clotting in the CAM blood test unit. The sealed CAM-blood tube may be placed on a roller and rotated at 5-30 cycles per minute at about 37° C., and then incubated for 1-3 hours for cell attachment to occur.

### EXAMPLE 2

### Specificity and Sensitivity Control

Human tumor cell lines of different tumor origins may be chosen for use in performing specificity and sensitivity control experiments. For examples, the human colon tumor cell line SW-480, human gastric tumor cell line RF-48, several breast tumor cell lines, human malignant melanoma line LOX, and several ovarian tumor cell lines may be used. Tumor cell lines may be purchased from American Type Culture Collection (Manassas, Va.). All cell lines should be confirmed to be negative for Mycoplasma infection. The tumor cell lines should be examined for: (a) high affinity binding to CAM within one hour after plating; (b) high proliferation rate; and (c) the tumor cell lines should be readily and stably (100%) fluorescently labeled with red or green fluorescent dyes prior to use or transformed with an expression plasmid for green fluorescent protein (GFP) in order to be able to visualize the tumor cells directly at the end of the enrichment procedures. Control normal blood will be seeded with known numbers of the green fluorescence labeled or GFP-expressing fluorescent human tumor cells and subjected to the CAM cell enrichment methods, to assess their comparable efficiencies.

Whole blood from a healthy donor or cord blood derived from umbilical cords may be obtained through the National Disease Research Interchange (Philadelphia). Immediately after reception, blood should be supplemented with Anticoagulant Citrate Dextrose solution USP (ACD, Baxter Healthcare Corporation, Deerfield, Ill.) plus lithium heparin to prevent clotting that often occurs during further experimental manipulations. Normal blood does not contain cells with cancer characteristics. Thus, the tumor cells spiked into these blood samples should be the only ones recovered in this test for specificity and sensitivity.

Cord blood or blood samples from healthy individuals may be seeded with known numbers of fluorescently-labeled, i.e., fluorescent dye pre-labeled or GFP-tagged tumor cells. The mixed blood samples of 3 mL aliquots may be transferred to CAM assay units for tumor cell enrichment. Suspended blood cells may be removed. When, for example, as type I collagen is the skeleton supporting the CAM film, the CAM-captured cells may be released into suspension using collagenase. To determine the number of control viable tumor cells from cord blood, for example, approximately 3,000 GFP-tumor cells may be spiked into 3 mL of cord blood (approximately 15,000,000,000 blood cells) or cell culture complete medium (containing 15% human serum) and subjected to CAM enrichment. Cells recovered from medium would indicate the number of actual viable tumor cells. The ratio, (cell number recovered from cord blood)/(cell number recovered from medium), signifies the efficiency of the assay. The percent recovery of viable tumor cells from cord blood as compared to medium may be used to determine optimal conditions for CAM enrichment assay. These conditions include period of time for incubation of CAM-blood tubes (e.g., 1-3 hours), rotation speed (e.g., 5-30 cycles per minute), and length of time of storing blood to retain cell viability (e.g., 4-48 hours). The presence of extremely large numbers of background blood cells would prevent direct contact of cancer cells with the CAM surface and diminish detection sensitivity of the CAM method. The CAM film of the blood collection tube advantageously is designed to maximize surface contact areas of CAM to tumor cells. Length of cell incubation time is also important, as CAM depends on differential adhesion of tumor cells than hematopoietic cells.

### EXAMPLE 3

### Determination of Cell Viability in a CAM-Blood Filtration Unit v. Blood Collection Tube

Another problem is the cell viability of the blood samples, which may vary during transportation to the research laboratory. Increasing the time of storage may be expected to damage cells in the blood. To determine if tumor cells in the CAM blood unit can stay viable during shipping, 3,000 GFP-tumor cells were spiked into 3 mL of cord blood and control medium containing 15% human serum (Sigma). Each aliquot was stored at 4° C. for series of time (4, 6, 8, 12, 16, 24, 36 and 48 hours). Each aliquot was then captured by CAM and the percent recovery of GFP-tumor cells by CAM determined. For each time point, four duplicate experiments were performed, and percent recoveries determined. The results showed that CAM-captured tumor cells survived better than suspended cells in blood.

CAM-enriched cells may be counted by any means known to those of ordinary skill in the art, including microscopic and flow cytometric methods (see below for detailed methods). For cell enrichment experiments, preliminary data obtained by microscopic counting suggest the recovery rate increases with spike dosage, roughly following a logistic curve. Using a CAM-initiated cell isolation device of the present disclosure, one can obtain approximately 40% recovery of the GFP-LOX human malignant melanoma cells spiked into cord blood when there is greater than 1,000 GFP-LOX cells per mL of blood in the initial sample, with a variability of approximately 10%.

Strategy for Enumeration and Validation of Viable Tumor Cells in Blood of a Subject by Flow Cytometry

In a clinical laboratory, labeled tumor cells can be measured by multi-parameter flow cytometric cell analyzer using FITC labeled collagen (green) to detect invasive tumor cells, PE labeled anti-CD45 leukocyte common antigen antibody (red) to detect and exclude leukocytes, and 7-MD to exclude dead cells. This automatic cellular analysis can be validated by a parallel and independent microscopic evaluation using microscopy, for example, with cell lineage markers including antibodies directed against epithelial, endothelial and hematopoietic antigens.

Enumeration of invasive tumor cells in blood by flow cytometry may be accomplished by multi-parameter flow cytometric cell analyzer using, for example: (a) FITC labeled collagen that would be ingested by tumor cells (green) to detect invasive tumor cells, and (b) PE-labeled anti-CD45 leukocyte common antigen antibody (red) to detect and exclude leukocytes contaminated in the cell population. For example, tumor cells captured by CAM and co-isolated normal blood cells may be post-stained with phycoerythrin (PE)-conjugated CD45 antibody and dead-cell nucleic acid dye 7-MD. Labeled cell sample may be aspirated and analyzed, for example, on a FACSCalibur flow cytometer (Becton Dickinson). Criteria for data analysis may include, among other factors: (a) size defined by forward light scatter, (b) granularity defined by orthogonal light scatter, (c) negative events of dead 7-AAD cells, (d) negative events of PE-labeled CD45 mAb normal cells, and (e) positive events of the FITC-tumor cells.

As would be understood by one of ordinary skill in the art, there are several cytometric methods of discriminating apoptotic and dead cells from alive cells in heterogeneous clinical specimens (e.g., using FITC-libeled annexin V and propidium iodide). For example, to incorporate the cell viability test into the multiparameter flow cytometry of CAM purified cells, one may use 7-amino-actinomycin D (7-AAD, Molecular Probes) to label dead cells in a fixed CAM cell population. 7-AAD can be excited by the 488 nm argon laser line and emits in the far red range of the spectrum. 7-AAD spectral emission can be separated from the emissions of FITC and PE (OLIVER et al., 1999). The fluorescence parameters allow characterization of dead cells (7-AAD), viable and invasive tumor cells (FITC-collagen) and leukocytes (PE-CD45) in a subset of CAM purified blood cells. Freshly labeled cells may be delivered to the flow lab for immediate counting or stored in suspension, for example, at 4° C. for 1-3 days. The FACSCalibur flow cytometer may be configured to count 2-4 cell samples per hour.

In a typical blood sample obtained from an individual with cancer or cardiovascular diseases, the circulating tumor and endothelial cells are vastly outnumbered (in the range of over a million-fold) by the normal hematopoietic cells.

While the embodiments described are not limited to any particular hypothesis, the present inventors postulate that:
(a) During the earliest stage of cancer progression, metastatic cells start emerging from primary tumors; these cells exhibit an invasive behavior,
(b) Tumor cell populations from blood that are indicative of the presence of a cancer will enable early diagnosis and further molecular analysis, and
(c) There are diagnostic sets of genes present in both circulating and primary tumor cells that can be used to: resolve the tissue-site origin of circulating tumor cells, determine a specific cancer subtype, and predict the metastatic potential of a patient with a high degree of confidence.
   Microscopic Characterization of the Cells Enriched by CAM Culture Method

A high yield, CAM culture may be performed in parallel as an independent CAM method to validate the tumor cells enriched by CAM and counted by flow cytometry. The CAM culture method can be readily augmented with microscopy and immunocytochemistry using cell lineage or putative tumor markers. Microscopy can be used to identify the CTC enriched from blood by CAM as possessing the following features denoted Co+/Epi+/Endo+/Leu−; the CEC as Co−/Epi−/Endo+/ Leu−; tumor-associated lymphocytes as Co−/Epi−/Endo−/Leu+. Specifically, the CTC are:
1) Positive fluorescence from ingested and concentrated TRITC-labeled collagen fragments (Co+; the proclivity to degrade and ingest ECM is one of the hallmarks of invasive and metastatic cells).
2) Positive immunocytochemical detection for the epithelial-specific markers, including cytokeratins and epithelial membrane antigens (BerEP4, EpCAM, GA733 and Muc-I) (Epi+).
3) Positive immunocytochemical detection for the endothelial specific markers, including CD31, van Willebrand factor (vWF) and VEGF receptor (Endo+).
4) Negative immunocytochemical detection for markers of the leukocyte/monocyte lineages, including CD45, CDI4 and CD68; negative for leukocyte-like cytology (Leu−).

The antibody labeling design of the CAM cell chamber method, in combination with differential interference contrast (DIC) bright field and use of a triple fluorescent filter, employable for example on a Nikon Eclipse E300 inverted fluorescent microscope, provide a powerful multiplex means of characterizing tumor cells in each microscopic field. In the same fluorescence microscopic field, TRITC-collagen labeling of invasive cells is seen as red fluorescence, FITC-cell type marker as green florescence and Hoechst 33258 nuclear dye as blue-fluorescence, whereas APAAP stained cell type marker is shown as red color in DIC bright light. Images may be stored in a computer hard drive and the number of color-or fluorescence-labeled cells in a sample may be counted with the aid of software such as Metamorph image analysis software (Universal Imaging Corporation).

Slides with the CAM-enriched and labeled cells may be scanned under fluorescent light microscopy for positive tumor cells.

Multiplex Molecular Analysis of CAM-Enriched Cells: Microarray and Real-Time RT-PCR

The expression levels of mRNAs expected to be present specifically in circulating tumor cells versus those expected to be present in leukocytes may be used as a measure of the degree to which enrichment is successful. The percentage of tumor cells in a given cell population may be validated using expression of epithelial (GA733-1) and leukocyte (CD45) markers, using tumor cell lines and leukocyte cell samples as positive controls.

Real-time RT-PCR may be performed using, for example, the Roche Light Cycler on cell samples purified from blood samples. Real-time PCR quantification of the epithelial marker GA733-1 and the leukocyte marker CD45 relative to β-actin may be performed. The epithelial marker GA733-1 is expected to be expressed at high levels in the pure tumor cell subsets and tumor cell lines but not in leukocytes. In turn, the leukocyte marker CD45 should be detected in the leukocyte samples and impure tumor cell populations but not in tumor cell lines nor in pure tumor cell samples. Observation of a substantial GA733-1 signal in the tumor cell sample recovered can be interpreted as demonstrating that the CAM enrichment procedure returns a cell pool in which tumor-characteristic markers can easily and reproducibly be measured. It is also important to determine the level of CD45 signal in each CAM tumor cell set to indicate degrees of contamination of leukocytes. If substantial contamination is observed, then one may conclude that, for example, a CD45 negative-selection step may be necessary to test and incorporate into the final protocol.

The molecular basis of most solid cancers is not understood. In each clinical specimen, carcinoma cells are variable in number and pathological types; carcinoma cells are also surrounded by numerous types and number of normal cells. Furthermore, tumor cells alter their gene expression profiles during progression and metastasis. The CAM cell enrichment methods offer viable tumor cell populations that are available for the molecular analysis of the tumor cells ex vivo using DNA microarray and real-time RT-PCR analyses. These viable tumor cell populations can enable a broad investigation into finding genes commonly expressed in the tumor cells derived from primary tumors and blood, and genes that are specifically expressed in the tumor cells of specific epithelial cancers. As seen in Table 1 and 2, the present cell separation method has allowed for the characterization of tumor cells isolated from blood samples using microarrays and RT-PCR technologies. The data show the characteristic gene expression for specific tumor cell types.

TABLE 1
Histo-pathological information of cell samples and their original clinical specimens *

| | | | | | | Micro- | RealTime |
|---|---|---|---|---|---|---|---|
| Category | Sample | Site | Histology | Grade | Stage | array | PCR |
| Tumor Cells | AO1 | Ovary | Serous adenocarcinoma | 3 | IIIC | v | v |
| from Ascites | AO2 | Ovary | Serous adenocarcinoma | 3 | IIIC | v | |
| | AO3 | Ovary | Serous adenocarcinoma | 3 | IIIC | v | v |
| | AO4 | Pimary | Serous adenocarcinoma | 3 | IIIC | v | v |
| | | peritoneal | | | | | |
| | AO5 | Ovary | Mixed clear cell, papillary and | 3 | IIIC | v | v |
| | | | endometrioid adenocarcinoma | | | | |
| | AO6 | Ovary | Serous adenocarcinoma | 3 | IIIC | | v |
| | AO7 | Ovary | Serous adenocarcinoma | 3 | IIIC | | v |
| | AO8 | Ovary | Serous adenocarcinoma | 3 | IIIC | | v |
| | AO9 | Ovary | Serous adenocarcinoma | 3 | IIIC | | v |
| | AO10 | Ovary | Serous adenocarcinoma | N/A | IIIC | | v |
| | AO11 | Ovary | Serous adenocarcinoma | 3 | IIIC | | v |
| | AO12 | Ovary | Serous adenocarcinoma | 3 | IIIC | | v |
| | AO13 | Ovary | Serous adenocarcinoma | 3 | IIIC | | v |
| | AO14 | Ovary | Serous adenocarcinoma | 3 | IIIC | | v |
| | AO15 | Primary | Serous adenocarcinoma | N/A | IIIC | | v |
| | | peritoneal | | | | | |
| | AO16 | Ovary | Clear cell adenocarcinoma | 3 | IIIC | | v |
| | AO17 | Ovary | Clear cell adenocarcinoma | 3 | IIIC | | v |
| | AO18 | Ovary | Clear cell adenocarcinoma | 3 | IIIC | | v |
| | AO19 | Ovary | Clear cell adenocarcinoma | 3 | IIIC | | v |
| | AO20 | Ovary | Clear cell adenocarcinoma | 3 | IIIC | | v |
| | AU1 | Endometrium | Serous adenocarcinoma | 3 | IVB | v | v |
| | AU2 | Endometrium | Serous adenocarcinoma | 3 | IVB | v | |
| | AU3 | Endometrium | Serous adenocarcinoma | 3 | IVB | v | v |
| | AU4 | Endometrium | Serous adenocarcinoma | 3 | IVB | v | v |
| | AU5 | Endometrium | Serous adenocarcinoma | 3 | IVB | v | v |
| | AU6 | Endometrium | Serous adenocarcinoma | 3 | IVB | | v |
| Cell Line | CL1 | OVCAR3 | | | | v | v |
| | CL2 | SKOV3 | | | | v | v |
| Tumor Cell | TO1 | Ovary | Serous adenocarcinoma | 3 | IIIC | v | v |
| from Primary | TO2 | Ovary | Serous adenocarcinoma | 3 | IV | v | v |
| Tumors | TO3 | Ovary | Serous adenocarcinoma | 3 | IIIC | v | v |
| | TO4 | Ovary | Serous adenocarcinoma | 3 | IIIC | v | v |
| | TO5 | Ovary | Serous adenocarcinoma | 3 | IIIC | | v |
| | TO6 | Ovary | Serous adenocarcinoma | 3 | IIIC | | v |
| | TO7 | Ovary | Serous adenocarcinoma | 3 | IIIC | | v |
| | TO8 | Ovary | Serous adenocarcinoma | 3 | IIIC | | v |
| | TO9 | Ovary | Serous adenocarcinoma | 3 | IIIC | | v |
| | TO10 | Ovary | Serous adenocarcinoma | 3 | IIIC | | v |
| | TG1 | Ovary | Granulosa | Adult- | IIC | v | |
| | | | | type | | | |
| | TG2 | Ovary | Granulosa | Adult- | IIC | v | v |
| | | | | type | | | |
| | TG3 | Ovary | Granulosa | Adult- | IIC | v | v |
| | | | | type | | | |
| | TG4 | Ovary | Granulosa | Adult- | IIC | v | v |
| | | | | type | | | |
| Fibroblasts | FB1 | Head & Neck | | | | v | |
| | FB2 | Head & Neck | | | | v | |
| | FB3 | Ovary | Fibroma | Benign | Benign | v | v |
| | FB4 | Ovary | Serous adenocarcinoma | 3 | IV | v | v |
| | FB5 | Ovary | Serous adenocarcinoma | 3 | IIIC | v | |
| | FB6 | Ovary | Mixed clear cell, papillary and | 3 | IIIC | v | v |
| | | | endometrioid adenocarcinoma | | | | |
| | FB7 | Ovary | Serous adenocarcinoma | 3 | IIIC | | v |
| | FB8 | Ovary | Serous adenocarcinoma | 3 | IIIC | | v |
| | FB9 | Ovary | Clear cell adenocarcinoma | 3 | IIIC | | v |
| Leukocytes | LE1 | Ovary | Serous adenocarcinoma | 3 | IV | v | |
| | LE2 | Ovary | Serous adenocarcinoma | 3 | IV | v | v |
| | LE3 | Ovary | Serous adenocarcinoma | 3 | IIIC | v | |
| | LE4 | Ovary | Serous adenocarcinoma | 3 | IIIC | v | |
| | LE5 | Ovary | Serous adenocarcinoma | 3 | IV | v | |
| | LE6 | Ovary | Serous adenocarcinoma | 3 | IIIC | v | v |
| | LE7 | Ovary | Serous adenocarcinoma | 3 | IV | v | v |
| | LE8 | Ovary | Serous adenocarcinoma | 3 | IIIC | v | |
| | LE9 | Ovary | Serous adenocarcinoma | 3 | IIIC | v | |
| | LE10 | Ovary | Serous adenocarcinoma | 3 | IIIC | v | |
| | LE11 | Ovary | Serous adenocarcinoma | 3 | IIIC | v | |
| | LE12 | Pimary | Serous adenocarcinoma | 3 | IIIC | v | v |
| | | peritoneal | | | | | |
| | LE13 | Pimary | Serous adenocarcinoma | 3 | IIIC | v | v |
| | | peritoneal | | | | | |
| | LE14 | Ovary | Mixed clear cell, papillary and | 3 | IIIC | v | v |
| | | | endometrioid adenocarcinoma | | | | |
| | LE15 | Endometrium | Serous adenocarcinoma | 3 | IVB | v | v |
| | LE16 | Ovary | Serous adenocarcinoma | 3 | IC | | v |
| | LE17 | Ovary | Serous adenocarcinoma | 3 | IC | | v |
| | LE18 | Ovary | Serous adenocarcinoma | 3 | IIIC | | v |
| | LE19 | Ovary | Serous adenocarcinoma | 3 | IIIC | | v |
| | LE20 | Ovary | Serous adenocarcinoma | 3 | IIIC | | v |
| | LE21 | Ovary | Serous adenocarcinoma | 3 | IIIC | | v |
| | LE22 | Ovary | Serous adenocarcinoma | 3 | IIIC | | v |
| | LE23 | Ovary | Serous adenocarcinoma | 3 | IIIC | | v |
| | LE24 | Primary | Serous adenocarcinoma | N/A | IIIC | | v |
| | | peritoneal | | | | | |
| | LE25 | Primary | Serous adenocarcinoma | 3 | IV | | v |
| | | peritoneal | | | | | |
| | LE26 | Primary | Serous adenocarcinoma | 3 | IV | | v |
| | | peritoneal | | | | | |

* Among the 77 total cell samples, 41 cell samples were examined by DNA microarray; 63 cell samples by real-time RT-PCR; 27 cell samples by both DNA microarray and real-time RT-PCR.

TABLE 2A
126 genes up-regulated in different types of tumor cells
enriched from ovarian and uterine tumor specimens

| Probe | Gene Bank | Common | Description | UniGene |
|---|---|---|---|---|
| 977_s_at | Z35402 | E-cadherin | H. sapiens gene encoding E-cadherin | |
| 38324_at | AD000684 | LISCH7 | liver-specific bHLH-Zip transcription | |
| | | | factor LISCH7 | |
| 575_s_at | M93036 | GA733-2 | GA733-2 | |
| 266_s_at | L33930 | CD24 | CD24 (small cell lung carcinoma | Hs.375108 |
| | | | cluster 4 antigen) | |
| 291_s_at | J04152 | M1S1 | GA733-1 | |
| 35276_at | AB000712 | hCPE-R | claudin 4 | Hs.5372 |
| 34674_at | X58079 | S100A1 | S100 calcium binding protein A1 | Hs.433503 |
| 35207_at | X76180 | SCNN1A | sodium channel, nonvoltage-gated 1 alpha | Hs.446415 |
| 33904_at | AB000714 | hRVP1 | claudin 3 | Hs.25640 |
| 32821_at | AI762213 | LCN2 | lipocalin 2 (oncogene 24p3) | Hs.204238 |
| 38783_at | J05581 | MCNAA | mucin 1, transmembrane | Hs.89603 |
| 700_s_at | | | mucin 1, transmembrane | |
| 38784_g_at | J05581 | MCNAA | mucin 1, transmembrane | Hs.89603 |
| 38482_at | AJ011497 | CLDN7 | claudin 7 | Hs.278562 |
| 2011_s_at | U34584 | BIK | BCL2-interacting killer (apoptosis- | Hs.155419 |
| | | | inducing) | |
| 37909_at | L34155 | LamA3 | laminin, alpha 3 | Hs.83450 |
| 38086_at | AB007935 | KIAA0466 | immunoglobulin superfamily, member 3 | Hs.81234 |
| 37483_at | AB018287 | KIAA0744 | histone deacetylase 9 | Hs.116753 |
| 33572_at | U78722 | Zpf165 | zinc finger protein 165 | Hs.55481 |
| 33282_at | U42408 | LAD | ladinin 1 | Hs.18141 |
| 39951_at | L20826 | PLS1 | plastin 1 (I isoform) | Hs.203637 |
| 36929_at | U17760 | LAMB3 | Homo sapiens laminin S B3 chain (LAM) gene | |
| 38051_at | X76220 | MAL | H. sapiens MAL gene exon 1 (and joined CDS). | |
| 34775_at | AF065388 | TSPAN-1 | tetraspan 1 | Hs.38972 |
| 36869_at | X69699 | PAX8 | paired box gene 8 | Hs.308061 |
| 33323_r_at | X57348 | | H. sapiens mRNA (clone 9112). | |
| 668_s_at | L22524 | MMP7 | Human matrilysin gene | |
| 41610_at | AB011105 | KIAA0533 | laminin, alpha 5 | Hs.11669 |
| 34348_at | U78095 | SPINT2 | serine protease inhibitor, Kunitz | Hs.31439 |
| | | | type, 2 | |
| 1898_at | L24203 | TRIM29 | tripartite motif-containing 29 | Hs.82237 |
| 40425_at | M57730 | B61 | ephrin-A1 | Hs.399713 |
| 34213_at | AB020676 | KIAA0869 | KIBRA protein | Hs.434243 |
| 927_s_at | J05582 | MUC1 | Human pancreatic mucin mRNA, complete cds. | |
| 41286_at | X77753 | TROP-2 | tumor-associated calcium signal | Hs.23582 |
| | | | transducer 2 | |
| 1585_at | M34309 | ERBB3 | v-erb-b2 erythroblastic leukemia | Hs.306251 |
| | | | viral oncogene homolog 3 (avian) | |
| 889_at | M73780 | ITGB8 | integrin, beta 8 | Hs.355722 |
| 863_g_at | U04313 | SERPINB5 | serine (or cysteine) proteinase | Hs.55279 |
| | | | inhibitor, clade B (ovalbumin), | |
| | | | member 5 | |
| 40218_at | U60808 | CDS | CDP-diacylglycerol synthase | Hs.380684 |
| | | | (phosphatidate cytidylyltransferase) 1 | |
| 35280_at | Z15008 | LAMC2 | laminin, gamma 2 | Hs.54451 |
| 41377_f_at | J05428 | UGT2B7 | UDP glycosyltransferase 2 family, | Hs.10319 |
| | | | polypeptide B7 | |
| 35148_at | AC005954 | TJP3 | Tight junction protein 3 | |
| 37286_at | AB002341 | KIAA0343 | neuronal cell adhesion molecule | Hs.7912 |
| 38489_at | M60047 | HBp17 | heparin-binding growth factor | Hs.1690 |
| | | | binding protein | |
| 40434_at | U97519 | PODXL | podocalyxin-like | Hs.16426 |
| 31792_at | M20560 | ANX3 | annexin A3 | Hs.442733 |
| 37920_at | U70370 | Bft | paired-like homeodomain transcription | Hs.84136 |
| | | | factor 1 | |
| 34771_at | AF035959 | PAP2-g | phosphatidic acid phosphatase type 2C | Hs.24879 |
| 36591_at | X06956 | TUBA1; | Human HALPHA44 gene for alpha-tubulin | |
| 330_s_at | | | Tubulin, alpha1, isoform 44 | |
| 41660_at | AL031588 | CELSR1 | Cadherin | |
| 36890_at | AF001691 | PPL | periplakin | Hs.192233 |
| 31610_at | U21049 | DD96 | membrane-associated protein 17 | Hs.431099 |
| 33128_s_at | W68521 | CST6 | cystatin E/M | Hs.139389 |
| 32139_at | Y09538 | ZNF185 | zinc finger protein 185 (LIM domain) | Hs.16622 |
| 41352_at | X62822 | SIAT1 | sialyltransferase 1 (beta-galactoside | Hs.2554 |
| | | | alpha-2,6-sialyltransferase) | |
| 33272_at | AA829286 | SAA1 | serum amyloid A1 | Hs.332053 |
| 408_at | X54489 | MGSA | Human gene for melanoma growth | |
| | | | stimulatory activity (MGSA). | |
| 35281_at | U31201 | LAMC2 | Human laminin gamma2 chain gene (LAMC2) | |
| 41376_i_at | J05428 | UGT2B7 | UDP glycosyltransferase 2 family, | Hs.10319 |
| | | | polypeptide B7 | |
| 40705_at | AF103905 | EPAC | Rap1 guanine-nucleotide-exchange | Hs.8578 |
| | | | factor directly activated by cAMP | |
| 35444_at | AC004030 | | Homo sapiens DNA from chromosome 19, | |
| | | | cosmid F21856 | |
| 1886_at | U53476 | Wnt7a | wingless-type MMTV integration site | Hs.72290 |
| | | | family, member 7A | |
| 40679_at | U27699 | SLC6A12 | solute carrier family 6 | Hs.82535 |
| | | | (neurotransmitter transporter, | |
| | | | betaine/GABA), member 12 | |
| 37533_r_at | D86980 | KIAA0227 | KIAA0227 protein | Hs.79170 |
| 35023_at | U00803 | FRK | fyn-related kinase | Hs.89426 |
| 36292_at | U07225 | P2RY2 | purinergic receptor P2Y, G-protein | Hs.339 |
| | | | coupled, 2 | |
| 40217_s_at | U65887 | CDS1 | CDP-diacylglycerol synthase | Hs.380684 |
| | | | (phosphatidate cytidylyltransferase) 1 | |
| 1887_g_at | U53476 | Wnt7a | wingless-type MMTV integration site | Hs.72290 |
| | | | family, member 7A | |
| 36105_at | M18728 | CEACAM6 | Carcinoma embryonic antigen-related | |
| | | | cell adhesion molecule 6 | |
| 39912_at | AB006179 | HS6ST1 | heparan sulfate 6-O-sulfotransferase 1 | Hs.380792 |
| 35577_at | AF027866 | SERPINB7 | serine (or cysteine) proteinase | Hs.138202 |
| | | | inhibitor, clade B (ovalbumin), | |
| | | | member 7 | |
| 40314 at | AJ002309 | SYNGR3 | synaptogyrin 3 | Hs.435277 |
| 142_at | U75308 | hTAFII130 | TAF4 RNA polymerase II, TATA box | Hs.24644 |
| | | | binding protein (TBP)-associated factor | |
| 41066_at | AF071219 | SCGB2A1 | secretoglobin, family 2A, member 1 | Hs.97644 |
| 39575_at | AF052143 | MOT8 | transmembrane protein SHREW 1 | Hs.25924 |
| 36010_at | U10492 | MOX1 | mesenchyme homeo box 1 | Hs.438 |
| 157_at | U65011 | PRAME | preferentially expressed antigen in | Hs.30743 |
| | | | melanoma | |
| 38515_at | X51801 | BMP7 | bone morphogenetic protein 7 | Hs.170195 |
| | | | (osteogenic protein 1) | |
| 32558_at | AB021868 | PIAS3 | protein inhibitor of activated STAT3 | Hs.435761 |
| 34703_f_at | AA151971 | | | |
| 32163_f_at | AA216639 | | | |
| 31885_at | M64572 | PTPN3 | protein tyrosine phosphatase, non- | Hs.405666 |
| | | | receptor type 3 | |
| 1812_s_at | | | | |
| 41587_g_at | AF075292 | FGF18 | fibroblast growth factor 18 | Hs.87191 |
| 39579_at | U89916 | CLDN10 | claudin 10 | Hs.26126 |
| 39016_r_at | L42611 | KRT6E | keratin 6E | Hs.446417 |
| 41790_at | AL031230 | ALDH5A1 | | |
| 39882_at | U66035 | DDP | translocase of inner mitochondrial | Hs.125565 |
| | | | membrane 8 homolog A (yeast) | |
| 40717_at | AB001928 | CTSL2 | cathepsin L2 | Hs.87417 |
| 40710_at | D86322 | CLGN | calmegin | Hs.86368 |
| 881_at | M35198 | ITGB6 | integrin, beta 6 | Hs.57664 |
| 1317_at | X70040 | RON | macrophage stimulating 1 receptor | Hs.2942 |
| 41544_at | AF059617 | SNK | serum-inducible kinase | Hs.398157 |
| 38882_r_at | AF096870 | EBBP | tripartite motif-containing 16 | Hs.241305 |
| 1177_at | | | Dna-binding protein Ap-2 | |
| 1603_g_at | L33881 | PRKCI | protein kinase C, iota | Hs.355476 |
| 1602_at | L33881 | PRKCI | protein kinase C, iota | Hs.355476 |
| 32262_at | AL049669 | CGI-01 | CGI-01 protein | Hs.19469 |
| 40069_at | AF051850 | SVIL | supervillin | Hs.163111 |
| 36909_at | X62048 | Wee1 Hu | WEE1 homolog (S. pombe) | Hs.249441 |
| 2017_s_at | M64349 | CCND1 | cyclin D1 (PRAD1: parathyroid | Hs.371468 |
| | | | adenomatosis 1) | |
| 39962_at | U59305 | CDC42BPA | CDC42 binding protein kinase alpha | Hs.18586 |
| | | | (DMPK-like) | |
| 38881_i_at | AF096870 | EBBP | tripartite motif-containing 16 | Hs.241305 |
| 41359_at | Z98265 | PKP3 | plakophilin 3 | Hs.26557 |
| 39556_at | M96803 | SPTBN1 | spectrin, beta, non-erythrocytic 1 | Hs.205401 |
| 37902_at | L13278 | CRYZ | crystallin, zeta (quinone reductase) | Hs.83114 |
| 35709_at | AF038172 | FLJ11149 | hypothetical protein FLJ11149 | Hs.37558 |
| 36849_at | U90920 | PARG1 | PTPL1-associated RhoGAP 1 | Hs.430919 |
| 35803_at | S82240 | RhoE | ras homolog gene family, member E | Hs.6838 |
| 182_at | U01062 | ITPR3 | inositol 1,4,5-triphosphate receptor, type 3 | Hs.77515 |
| 37199_at | AI760932 | CGI-60 | dynein 2 light intermediate chain | Hs.309257 |
| 37832_at | AL080062 | DKFZp564I122 | DKFZP564I122 protein | Hs.13024 |
| 168_at | U50196 | ADK | adenosine kinase | Hs.355533 |
| 37728_r_at | X78669 | ERC-55 | reticulocalbin 2, EF-hand calcium | Hs.79088 |
| | | | binding domain | |
| 41060_at | M74093 | CCNE1 | cyclin E1 | Hs.244723 |
| 38007_at | L11353 | NF2 | neurofibromin 2 (bilateral acoustic | Hs.902 |
| | | | neuroma) | |
| 41781_at | U22815 | PPFIA1 | protein tyrosine phosphatase, | Hs.128312 |
| | | | receptor type, f polypeptide (PTPRF), | |
| | | | interacting protein (liprin), alpha 1 | |
| 38340 at | AB014555 | KIAA0655 | huntingtin interacting protein-1-related | Hs.96731 |
| 40004_at | X91868 | six1 | sine oculis homeobox homolog 1 | Hs.54416 |
| | | | (Drosophila) | |
| 37143_s_at | AB002359 | KIAA0361 | phosphoribosylformylglycinamidine | Hs.88139 |
| | | | synthase (FGAR amidotransferase) | |
| 34189_at | D31891 | KIAA0067 | SET domain, bifurcated 1 | Hs.345058 |
| 40762_g_at | AA705628 | SLC16A5 | solute carrier family 16 | Hs.90911 |
| | | | (monocarboxylic acid transporters), | |
| | | | member 5 | |
| 41294_at | AJ238246 | SCL | Homo sapiens mRNA for sarcolectin. | |
| 35766_at | M26326 | KRT18 | keratin 18 | Hs.406013 |
| 40445_at | AF017307 | ERT | E74-like factor 3 (ets domain | Hs.67928 |
| | | | transcription factor, epithelial-specific) | |
| 1681_at | X03635 | ESR1 | estrogen receptor 1 | |

TABLE 2B
48 genes up-regulated in different types of leukocytes
enriched from ovarian and uterine tumor specimens

| Probe | Gene Bank | Common | Description | UniGene |
|---|---|---|---|---|
| 931_at | L08177 | EBI2 | lymphocyte-specific G protein-coupled receptor | Hs.784 |
| 40520_g_at | Y00638 | PTPRC | CD 45, protein tyrosine phosphatase, receptor | Hs.444324 |
| | | | type, C | |
| 40742_at | M16591 | HCK | protein-tyrosine kinase; Human | |
| | | | hemopoietic cell protein-tyrosine | |
| | | | kinase (HCK) gene | |
| 38533_s_at | J03925 | ITGAM | integrin, alpha M | Hs.172631 |
| 35659_at | U00672 | IL10RA | interleukin 10 receptor, alpha | Hs.327 |
| 33641_g_at | Y14768 | AIF1 | allograft inflammatory factor 1 | |
| 35261_at | W07033 | GMFG | glia maturation factor, gamma | Hs.5210 |
| 40019_at | M60830 | EVI2B | open reading frame; Human EVI2B3P gene | |
| 38796_at | X03084 | C1QB | complement component 1, q | Hs.8986 |
| | | | subcomponent, beta polypeptide | |
| 37975_at | X04011 | CYBB | cytochrome b-245, beta polypeptide | Hs.88974 |
| 37011_at | U49392 | AIF-1 | allograft inflammatory factor 1 | Hs.76364 |
| 39994_at | D10925 | HM145 | chemokine (C—C motif) receptor 1 | Hs.301921 |
| 34660_at | AI142565 | RNASE6 | ribonuclease, RNase A family, k6 | Hs.23262 |
| 35012_at | M81750 | MNDA | myeloid cell nuclear differentiation antigen | Hs.153837 |
| 39221_at | AF004231 | MIR cl-10 | leukocyte immunoglobulin-like | Hs.306230 |
| | | | receptor, subfamily B, member 3 | |
| 37220_at | M63835 | CD64 | Human IgG Fc receptor I gene, exon | |
| | | | 6 and complete cds. | |
| 34210_at | N90866 | CDW52 | CDW52 antigen (CAMPATH-1 antigen) | Hs.276770 |
| 38363_at | W60864 | TYROBP | TYRO protein tyrosine kinase binding | Hs.9963 |
| | | | protein | |
| 35926_s_at | AF004230 | MIR cl-7 | leukocyte immunoglobulin-like | Hs.149924 |
| | | | receptor, subfamily B, member 1 | |
| 36889_at | M33195 | FCER1G | Fc fragment of IgE, high affinity I, | Hs.433300 |
| | | | receptor for; gamma polypeptide | |
| 37759_at | U51240 | LAPTm5 | Lysosomal-associated multispanning | Hs.436200 |
| | | | membrane protein-5 | |
| 31870_at | X14046 | CD37 | CD37 antigen | Hs.153053 |
| 40519_at | Y00638 | PTPRC | CD45, protein tyrosine phosphatase, | Hs.444324 |
| | | | receptor type, C | |
| 40518_at | Y00062 | PTPRC | CD45, protein tyrosine phosphatase, | Hs.444324 |
| | | | receptor type, C | |
| 40331_at | AF035819 | MARCO | macrophage receptor with collagenous structure | Hs.67726 |
| 37918_at | M15395 | LYAM1 | integrin, beta 2 | Hs.375957 |
| 32068_at | U62027 | HNFAG09 | complement component 3a receptor 1 | Hs.155935 |
| 39982_r_at | D13265 | MSR1 | macrophage scavenger receptor 1 | Hs.436887 |
| 36908_at | M93221 | MRC1 | Human macrophage mannose receptor (MRC1) gene | |
| 31499_s_at | X16863 | FCGR3B | Fc fragment of IgG, low affinity | Hs.372679 |
| | | | IIIa, receptor for (CD16) | |
| 37688_f_at | M31932 | FCGR2A | Fc fragment of IgG, low affinity | Hs.352642 |
| | | | IIa, receptor for (CD32) | |
| 37148_at | AF025533 | LIR-3 | leukocyte immunoglobulin-like | Hs.306230 |
| | | | receptor, subfamily B, member 3 | |
| 34223_at | M59818 | G-CSFR-1 | colony stimulating factor 3 receptor | Hs.381027 |
| | | | (granulocyte) | |
| 39319_at | U20158 | LCP2 | lymphocyte cytosolic protein 2 | Hs.2488 |
| 39857_at | AF044309 | STX11 | syntaxin 11 | Hs.118958 |
| 36879_at | M63193 | ECGF1 | endothelial cell growth factor 1 | Hs.435067 |
| | | | (platelet-derived) | |
| 1665_s_at | | | Interleukin 18 | |
| 33731_at | AJ130718 | y+LAT1 | solute carrier family 7 (cationic amino acid | Hs.194693 |
| | | | transporter, y+ system), member 7 | |
| 39593_at | AI432401 | FGL2 | fibrinogen-like 2 | Hs.351808 |
| 37541_at | U25956 | SELPLG | Human P-selectin glycoprotein ligand | |
| | | | (SELPLG) gene | |
| 37099_at | AI806222 | ALOX5AP | arachidonate 5-lipoxygenase- | Hs.100194 |
| | | | activating protein | |
| 38006_at | M37766 | MEM-102 | CD48 antigen (B-cell membrane protein) | Hs.901 |
| 41723_s_at | M32578 | HLA-DRB1 | major histocompatibility complex, | Hs.308026 |
| | | | class II, DR beta 3 | |
| 37039_at | J00194 | HLA-DRA | major histocompatibility complex, | Hs.409805 |
| | | | class II, DR alpha | |
| 35016_at | M13560 | CD74 | Ia-associated gamma chain; Human | |
| | | | Ia-associated invariant gamma-chain gene | |
| 38833_at | X00457 | HLA-DPA1 | major histocompatibility complex, | Hs.914 |
| | | | class II, DP alpha 1 | |
| 33374_at | L09708 | C2 | complement component 2 (C2) gene allele b, | |
| | | | exons 10 through 18 and complete cds. | |
| 36878_f_at | M60028 | HLA-DQB1 | major histocompatibility complex, | Hs.409934 |
| | | | class II, DQ beta 1 | |

TABLE 2C
45 genes up-regulated in different types of fibroblasts
enriched from ovarian and uterine tumor specimens

| Probe | Gene Bank | Common | Description | UniGene |
|---|---|---|---|---|
| 672_at | J03764 | PAI1 | plasminogen activator inhibitor-1 | |
| 1968_g_at | X76079 | PDGFRA | platelet-derived growth factor receptor, | Hs.74615 |
| | | | alpha polypeptide | |
| 659_g_at | L12350 | THBS2 | thrombospondin 2 | Hs.108623 |
| 658_at | L12350 | THBS2 | thrombospondin 2 | Hs.108623 |
| 37671_at | S78569 | laminin | laminin, alpha 4 | Hs.437536 |
| | | alpha 4 chain | | |
| 39945_at | U09278 | Seprase/FAPalpha | Seprase, FAP alpha | Hs.436852 |
| 38420_at | Y14690 | COL5A2 | collagen, type V, alpha 2 | Hs.283393 |
| 1466_s_at | S81661 | KGF | fibroblast growth factor 7 | Hs.374988 |
| 32307_s_at | V00503 | COL1A2 | collagen, type I, alpha 2 | Hs.232115 |
| 32306_g_at | J03464 | COL1A2 | collagen, type I, alpha 2 | Hs.232115 |
| 32305_at | J03464 | COL1A2 | collagen, type I, alpha 2 | Hs.232115 |
| 38637_at | L16895 | LOX | Human lysyl oxidase | |
| 36976_at | D21255 | osf-4 | cadherin 11 , type 2, | Hs.443435 |
| 2087_s_at | D21254 | osf-4 | cadherin 11 , type 2 | Hs.443435 |
| 36073_at | U35139 | NDN | necdin homolog | Hs.50130 |
| 1147_at | | | V-Erba Related Ear-3 Protein | |
| 32551_at | U03877 | S1-5 | EGF-containing fibulin-like extra- | Hs.76224 |
| | | | cellular matrix protein 1 | |
| 1731_at | M21574 | PDGFRA | platelet-derived growth factor | Hs.74615 |
| | | | receptor, alpha polypeptide | |
| 36233_at | AF091242 | PAPSS2 | S'-phosphoadenosine 5'-phosphosulfate | Hs.274230 |
| | | | synthase 2 | |
| 32488_at | X14420 | COL3A1 | collagen, type III, alpha 1 | Hs.443625 |
| 35234_at | D50406 | ST15 | reversion-inducing-cysteine-rich protein | Hs.388918 |
| | | | with kazal motifs | |
| 34303_at | AL049949 | FLJ90798 | hypothetical protein FLJ90798 | Hs.28264 |
| 33440_at | U19969 | TCF8 | Human two-handed zinc finger protein | |
| | | | ZEB mRNA | |
| 159_at | U43142 | VEGFC | vascular endothelial growth factor C | Hs.79141 |
| 456_at | U66619 | BAF60C | SWI/SNF related, matrix associated, | Hs.444445 |
| | | | actin dependent regulator of chromatin, | |
| | | | subfamily d, member 3 | |
| 33883_at | AB001466 | EFS | embryonal Fyn-associated substrate | Hs.24587 |
| 39395_at | AA704137 | THY1 | Thy-1 cell surface antigen | Hs.134643 |
| 39260_at | U59185 | MCT | solute carrier family 16 (monocarboxylic | Hs.351306 |
| | | | acid transporters), member 4 | |
| 33240_at | AB029018 | KIAA1095 | likely ortholog of mouse semaF | Hs.177635 |
| | | | cytoplasmic domain associated protein 3 | |
| 35347_at | AF093119 | UPH1 | EGF-containing fibulin-like extra- | Hs.381870 |
| | | | cellular matrix protein 2 | |
| 39069_at | AF053944 | AEBP1 | AE binding protein 1 | Hs.439463 |
| 581_at | M61916 | LAMB1 | laminin, beta 1 | Hs.122645 |
| 37578_at | D25248 | AFAP | actin filament associated protein | Hs.115912 |
| 33328_at | W28612 | HEG | 49b3 Human retina cDNA randomly primed | Hs.433452 |
| | | | sublibrary | |
| 1934_s_at | X94216 | VEGF-C | vascular endothelial growth factor C | Hs.79141 |
| 35366_at | M30269 | NID | nidogen (enactin) | Hs.356624 |
| 31897_at | U53445 | Doc1 | downregulated in ovarian cancer 1 | Hs.15432 |
| 35832_at | AB029000 | KIAA1077 | sulfatase 1 | Hs.409602 |
| 35985_at | AB023137 | KIAA0920 | A kinase (PRKA) anchor protein 2 | Hs.42322 |
| 36065_at | AF052389 | LDB1 | LIM domain binding 2 | Hs.4980 |
| 39973_at | U47926 | LEPREL2 | leprecan-like 2 protein | Hs.46458 |
| 32565_at | U66619 | BAF60C | SWI/SNF related, matrix associated, | Hs.444445 |
| | | | actin dependent regulator of | |
| | | | chromatin, subfamily d, member 3 | |
| 1319_at | X74764 | TKT | discoidin domain receptor family, | Hs.71891 |
| | | | member 2 | |
| 1834_at | D38449 | GPR | putative G protein coupled receptor | Hs.37196 |
| 35740_at | AL050138 | DKFZp586M121 | elastin microfibril interfacer 1 | Hs.63348 |

Methods and Compositions for the Determination of Host Immunity Against Tumor

Most CTC are dead or apoptotic in the circulation due to the presence of host immunity to tumors, as described in co-pending PCT Patent Application PCT/US01/26735. The CAM-initiated blood device, the viability of CTC, and the plasma derived from individual donors put together an effective means of determining host immunity against tumor. CAM-enriched CTC often form clusters with cytotoxic leukocytes. The cell-adhesion matrix could readily isolate such clusters of immune and cancer cell complex from patients who might exhibit encouraging prognosis. Furthermore, soluble components of complement system involving in tumor cytolysis could be determined by the viability of CTC in the presence of autologous plasma, derived from the blood of the same subject. Thus, the presence of tumor cytotoxic leukocytes and soluble complement system would be an important indicator for host immunity.

To determine the number of viable CTC in the presence of anti-tumor cytotoxic leukocytes and complement system, whole blood or the mononuclear cells in the presence of 10-20% autologous plasma may be screened by way of a CAM-initiated cell isolation device. When the number of CTC enriched by CAM is high in the absence of autologous plasma but low in the presence of autologous plasma, the subject could be high in anti-tumor immunity. On the other hand, high levels of viable CTC that resist immune killing detected in the presence and absence of autologous plasma would be the strongest indicator for patients who possess a high degree of malignancy.

### EXAMPLE 4

### CAM Positive Isolation of Tumor Cells from Whole Blood

An exemplar protocol that might be practiced for the isolation of tumor cells from whole blood is set forth below:
1. Preparation of cord blood: Add 3 mL of anticoagulated cord blood (plus 300 μg of ACD and lithium heparin) spiked with a known number of GFP-tumor cells into each tube of the CAM blood test unit. Place the sealed CAM-blood tube on a roller and rotate at 5-30 cycles per minute at 37° C. Incubate for 1-3 hours for tumor cell attachment to occur.
2. Preparation of control medium: Add 3 mL of control medium (plus 300 μl of ACD and lithium heparin) spiked with a known number of GFP-tumor cells into each tube of the CAM blood test unit. Place the sealed CAM-tumor tube on a roller and rotating at 5-30 cycles per minute at 37° C. Incubate for 1-3 hours for tumor cell attachment to occur.
3. Remove blood or medium supernatants carefully by pipetting. Wash the tubes five times in 3 mL without disturbing the CAM film on the inner wall Washing solution (PBS/0.1%, BSA/10%, ACD and lithium heparin).
4. Add 1 mL of collagenase solution into each tube of CAM blood filtration unit that has been thoroughly washed and clear of red cells. Place the sealed CAM-blood tube on the roller and rotate at 5 cycles per minute at 37° C. Incubate for 10 minutes, in order to dissolve CAM and release tumor cells into suspension. Collagenase solution (PBS, 0.3 mM CaCl₂, 0.2 μg/mL type I collagenase [Worthington Biochemical], 25 μg/mL DNase [Roche]).
5. Transfer the suspension to a new Bppendorf tube. Keep on ice for immediate immunofluorescent labeling using TRITC- anti-CD45 for microscopy or PE-anti-CD45 for flow cytometry. Labeled tumor cells will be counted by both microscopy and flow cytometry.

### EXAMPLE 5

### Fluorescent Material Containing CAM Film

As invadopodic cells digest and internalize ECM matrix, if the CAM matrix is fluorescent, then the tumor cells should become fluorescent during the enrichment process. To accomplish this, fluorescent TRITC or FITC-type I collagen polymers are incorporated into the CAM substrate before it is coated on the capture vessels. A negative identification procedure may be used to distinguish the cancer cells from leukocytes using phycoerythrmn (PE)- or FITC or TRITC-conjugated antibodies directed against the leukocyte common antigen CD45.

Currently, RT-PCR and immunocytochemistry (targeted against epithelial molecules, such as CK18 and CK20 cytokeratins, GA733 epithelial membrane antigens, Muc-1, and pan-epithelial antigen BerEP4) are used for confirmation of the epithelial origin of circulating tumor cells (Ghossein et al., 1999; Molnar et al., 2001; Racila et al., 1998; Schoenfeld et at, 1997; Soeth et al., 1997; Vlems et at, 2002; Wharton et al., 1999). Although both methods have high detection sensitivity and have successfully been used to resolve circulating tumor cells in blood after differential centrifugation enrichment (approximately 500) of the mononuclear cell fraction from whole blood, the detection rate remains low because circulating tumor cells represent less than 100 cells per one billion of normal cells in blood. In addition, it is not known if this approach captures the most critical cells, since genes responsible for metastatic progression to the circulation remain unknown. The use of anti-epithelial antibodies-based affinity purification would result in significant loss of tumor cells in blood.

In contrast, a one million-fold cell enrichment of CAM, which may be performed in one step, may achieve greater than 40% recovery of the 3,000 viable tumor cells from 15×10⁹ blood cells.

To further improve enrichment of the targeted cells, a multi-step cell enrichment procedure may be employed to recover greater than 85% of tumor cells from blood. This method involves first a density gradient centrifugation of whole blood cells to concentrate mononuclear cells, followed by culturing these cells on the fluorescent CAM film for an appropriate period of time, e.g., 12-18 hours, in order to: (a) label the tumor cells, (b) culture the tumor cells and less than 0.1% of leukocytes on CAM films, and (c) stain the CAM-captured cell population with antibodies or nucleic acid dyes. Both individual tumor cells and clumps may be readily observed by microscopy (whereas cell clumps often generate difficulty in flow cytometry).

A CAM blood filtration assay may be used to isolate viable tumor cells, endothelial progenitor cells and immune lymphocytes in the blood of patients with cancers. CAM-captured cells will then be seeded in parallel onto a 16-well chamber slide (Lab-Tek, Rochester, N.Y.) coated with FITC (or TRITC)-collagen-based CAM and cultured for 12-18 hours. Invasive tumor cells will ingest fluorescent CAM and become labeled with FITC (or TRITC), whereas co-purified endothelial cells and leukocytes will remain unlabeled. In addition to the positive identification of circulating tumor cells, isolated cells will be tested for a negative identification by labeling TRITC (or FITC)-CD45 or CD31 for fluorescent microscopy or with PE-CD45 or CD31 for flow cytometry.

### EXAMPLE 6

### Enumeration of Isolated Cells by Flow Cvtometry

Approximately 10 to 20 mL of blood per patient may be collected in Vacutainer tubes (Becton Dickinson, green top, lithium heparin anticoagulant, each tube holds 7-ml). Aliquots of freshly collected blood samples may be transferred to CAM blood test tubes or undergoing density gradient centrifugation to obtain the mononuclear cells, and subjected to further cell enrichment and identification on CAM. Enumeration of viable tumor cells in blood by flow cytometry may be accomplished based on following criteria: (a) tumors cells visualized via their ingestion of FITC labeled collagen; (b) PE-labeling of normal blood cells may be used as a complementary signal to identify contaminating leukocytes; (c) negative events of dead 7-AAD cells.

FITC-collagen- or GFP-tagged tumor cells may be captured by CAM and coisolated normal blood cells may be post-immuno-stained with phycoerytbrin (PE)-conjugated CD45 antibody. As little as a 500 μl sample may be aspirated and analyzed on a FACSCalibur flow cytometer (Becton Dickinson). Data may be acquired in listmode by using a threshold on the fluorescence of the nucleic acid dye 7-AAD. Criteria for multi-parameter data analysis include: (a) size defined by forward light scatter, (b) granularity defined by orthogonal light scatter, (c) negative events of dead 7-AAD cells, (d) positive events of the FITC-collagen- or GFP-tumor cells, and (e) negative events of PE-labeled CD45 mAb normal cells.

To enable the enumeration of tumor cells present in blood at frequencies below published rates of 100,000 tumor cells in 10,000,000,000 blood cells per mL of blood (Glaves et al., 1988; Karczewski et al., 1994) by flow cytometry, the following may be advantageously noted:
(i) The sample volume may be reduced from 3-20 mL to 500 μl and total cell count from 15,000,000,000 to 1,000,000 without a significant loss of tumor cells passing through the flow cytometer in a reasonable time period (sample flow rate=60 μl/mm).
(ii) The enriched tumor cells have to be distinguishable from normal cells co-isolated with them. The tumor cells may be FITC-collagen- or GFP-labeled, whereas more than 99% of the co-isolated cells should be leukocytes and may be labeled with phycoerytbrin (PE)-conjugated anti-CD45 antibody.
(iii) Tumor cells often may exist as clumps of 50 μm to 500 μm in diameter. Cell samples derived from the CAM blood filtration and antibody-based magnetic bead methods may be filtered through 50 μm mesh to remove large clumps before loading into the flow cytometer. Alternatively, when clumps are cultured on the CAM, circulating tumor cells break out from clumps and start to invade CAM films within 12-18 hours. When fluorescent CAM films are used, tumor cells enriched by the CAM method may be labeled with fluorescent collagen and they may be suspended by collagenases as individual cells.

### EXAMPLE 7

### CAM Enrichment of Tumor Cells from a Subject for use in Flow Cytometry

(1). Add 3 mL of anticoagulated blood (0.3 mL of lithium heparin plus Anticoagulant Citrate Dextrose solution USP-ACD, Baxter Healthcare Corporation, Deerfield, Jib) into each tube of the CAM blood filtration unit coated with FITC-labeled collagen. Place the sealed CAM-blood tube on a roller and rotate at 5-30 cycles per minute at 37° C. Incubate for 1-3 hours for tumor cell attachment to occur.

(2). Remove non-adherent cells and supernatants carefully by pipetting. Wash the tube five times in 3 mL solution carefully to avoid disturbing the CAM film on the inner wall. Washing solution (PBS/O 1% BSA 1% ACD and lithium heparin).

(3). Add 1 mL of the complete cell culture medium containing 15% human serum in HEPE buffer, pH 7.4 into each CAM blood filtration unit. Place the sealed CAM-blood tube on a roller and rotate at 5 cycles per minute at 37° C. Incubate for 9-15 hours to allow labeling of tumor cells with ingested FITC-type I collagen.

(4). Remove medium supernatants carefully by pipetting. Wash the tubes 3 times in 3 mL PBS without disturbing the CAM film on the inner wall.

(5). Add 1 mL of collagenase solution into each tube of CAM blood filtration unit that has been thoroughly washed. Place the sealed CAM-blood tube on the roller and rotate at 5 cycles per minute at 37° C. Incubate for 10 minutes, in order to dissolve CAM and release tumor cells into suspension. Collagenase solution (PBS, 0.3 mM CaCl₂, 0.2 μg/mL type I collagenase [Worthington Biochemical], 25 μg/mL DNase [Roche]).

(6). Transfer the suspension, 500 μl each, to one of two Eppendorf tubes.

(7). Staining/preparation for multi-parameter flow cytometry: Add 100 μl of fixative solution (PBS, 6% paraformaldehyde, pH 7.2) into the 500 μl cell suspension in an Eppendorf tube (final fixative concentration at 1% paraformaldehyde) and fix at 20-25 C for 10 minutes.

(8). Spin down cell pellet a t 1,000 rpm for 1 minute.

Remove fixative and wash the tube 3 times in 500 μl PBS solution. Keep on ice and add 10 μg/mL of PE-anti-CD45 (for marking leukocytes) and 1 μg/mL of 7-AAD (for staining dead cells), followed by incubation for 10 mm at 4° C. in the dark.

The protocol above is specified for CTC detection. For the detection of CEC and tumor-associated lymphocytes, PE-anti-CD31 and PE-anti-CD45 could be used to mark CEC and tumor-associated lymphocytes, respectively.

### EXAMPEL 8

### Tumor Cells Enriched by the CAM 96-Well Cell Chamber Method for use in Flow Cytometry

(1). Preparation of the MNC fraction by density centrifugation: Use remaining 3-15 mL of anticoagulated blood in a Vacutainer blood collection tube (Becton Dickinson, green top, lithium heparin as anticoagulant, each tube holds 7-mL). The cell pellet is spun down at 1,000 rpm and the cells are resuspended in 5 mL PBS containing 0.5 mM EDTA. The mononucleate cell (MNC) fraction is obtained by Ficoll-Paque density centrifugation (Pharmacia) according to manufacturer's instruction, washed in complete culture medium containing 15% bovine serum, and suspended in 3-15 mL of the complete medium.

(2). Culture of the MNC fraction on a CAM 96-well chamber slide: Seed 100 μl/well of the cell suspension (also applicable to the cells captured by other methods such as CAM and Dynal AAMB) onto desired wells, such 8 wells of a 96-well microtiter plate that were coated with FITC-collagen-based CAM that have been filled with 100 μl of complete culture medium containing 15% bovine serum and cultured in a CO2 incubator at 37° C. for 12-18 hours. This step labels tumor cells by assaying their ability to digest and internalize fluorescent collagen fragments.

(3). Non-adherent cells and supernatants are removed carefully by pipetting, and the wells are washed 2 times in 200 μl of PBS without disturbing the CAM film on the inner wall. Non-adherent cells consist of dead tumor cells and non-tumor blood cells in the MNC fraction. Suspended cells can be pooled and subjected to cell isolation for CD 19 leukocytes or stem cells.

(4). Add 100 μl of collagenase solution (PBS, 0.3 mM CaCl₂, 0.2 μg/mL type I collagenase [Worthington Biochemical], 25 μg/mL DNase [Roche]) into each well of the 8-well row of the 96-well CAM blood unit that has been thoroughly washed. The adherent cells are Incubate for 10 minutes, in order to dissolve CAM and release bound tumor cells into suspension.

(5). Transfer the suspension from the 8-well, 800 μl total, to Eppendorf tubes.

(6). Add 200 μl of fixative solution (PBS, 10% paraformaldehyde, pH 7.2) into the 800 μl cell suspension in an Eppendorf tube (final fixative concentration at 2% paraformaldehyde) and fix at 20-25° C. for 10 minutes.

(7). Spin down cell pellet at 1,000 rpm for 1 minute, remove the fixative and wash the tube 3 times in 500 μl PBS solution.

Keep cell pellet on ice and add 10 pg/mL of PE-anti-CD45, CD 14 and CD68 (for marking leukocytes, monocytes, macrophages) and 1 μg/mL of 7-AAD (for staining dead cells), followed by incubation for 10 minutes at 4° C. in the dark.

The protocol above is specified for CTC detection. For the detection of CEC and tumor-associated lymphocytes, PE-anti-CD31 and PE-anti-CD45 could be used to mark CEC and tumor-associated lymphocytes, respectively.

### EXAMPLE 9

### Microscopic Characterization of Tumor Cells Enriched by the CAM 16-Well Cell Chamber Method

(1) Preparation of the cellular and plasma fractions by low speed. 750 rpm for 5 mm, centrifugation: Spin down cell pellet in 3-7 mL of anticoagulated blood in a Vacutainer blood collection tube (Becton Dickinson, green top, lithium heparin as anticoagulant, each tube holds 7-ml) at 750 rpm for 5 mm or 1,000 rpm for 3 mm. Transfer the plasma from the supernatant of the centrifuged blood, 120 μl total, to an Eppendorf tube that are filled with 680 μl of anticoagulated complete culture medium containing 15% bovine serum [called the plasma medium: 15% plasma from a specific donor, in 10% anticoagulant (ACD and lithium heparmn) and 75% complete culture medium]. The rest of plasma is stored in 0.5 μL aliquots.

(2) Preparation of the M7NC fraction by density centrifugation: Cells will be resuspended in 5 mL PBS containing 0.5 mM EDTA. Mononucleate cell (MINC) fraction are obtained by Ficoll-Paque density centrifugation (Pharmacia) according to manufacturer's instruction, washed in complete culture medium containing 15% bovine serum, and suspended in same volume of the complete medium as blood prior to fractionation.

(3) Preparation of a CAM 16-well chamber slide pre-incubated with complete culture media with and without 15% plasma from each specific donor: Into each well of the upper 8-wells of a 16-well chamber slide (in 96-well microtiter plate format; Lab-Tek, Rochester, NY) coated with TRITC-collagen-based CAM, seed 100 μl of the complete culture medium and 10% anticoagulant. Into each well of the lower 8-wells of a 16-well chamber slide (in 96-well microtiter plate format; Lab-Tek, Rochester, NY) coated with TRITC collagen-based CAM, seed 100 μl of the complete culture medium and 10% anticoagulant, and 15% individual plasma [the plasma medium 15% plasma from a specific donor, in 10% anticoagulant (CDA+heparin), prepared in procedure 1].

(4) Culture of the MNC fraction on a CAM 16-well chamber slide: Seed 100 μl of the cell suspension (also applicable to the cells captured by other methods such as CAM and Dynal AAMB) onto each well of a 16-well chamber slide (in 96-well microtiter plate format; Lab-Tek, Rochester, N.Y.) coated with TRITC-collagen-based CAM that have been filled with 100 μl of complete culture medium containing 15% bovine serum and cultured in a CO₂ incubator at 37° C. for 12-18 hours. This step labels tumor cells by assaying their ability to digest and internalize fluorescent collagen fragments.

(5) Non-adherent cells and supernatants are removed carefully by pipetting. Non-adherent cells consist of dead tumor cells and non-tumor blood cells in the MNC fraction.

(6) Antibody and nucleic acid staining: Add 200 μl of fixative solution (PBS, 3.7% paraformaldehyde, pH 7.2) into each well of CAM labeling chamber unit and incubate at 20-25° C. for 10 minutes. The fixative is removed and cells in the wells are washed 3 times in 200 μl of PBS solution and kept on ice for immediate immuno-labeling using blue-fluorescent Hoechst 33342 nuclear dye and green-fluorescent FITC-anti-von Willebrand factor (marking an endothelial phenotype) for fluorescent microscopy, and red-color APMP- anti-ESA (cytokeratins, EMA etc epithelial markers, hematopoietic cell markers CD45/CD14/CD68/CDI9/CD8, or other endothelial cell markers CD31, fit-1, etc.) for DIC bright field microscopy.

The protocol above is specified for CTC detection. For the detection of CEC and tumor-associated lymphocytes, anti-CD31 and anti-CD45 could be used to mark CEC and tumor-associated lymphocytes, respectively, and then used to generate cRNA probes.

### EXAMPLE 10

### Tumor Cells Enriched by the CAM 96-Well Cell Chamber Method for use in Real-Time RT-PCR and DNA Microarray Molecular Analyses

(1) Preparation of the MNC fraction by density centrifugation [Parallel to Example 7 Protocol above]: Use remaining 3-15 mL of anticoagulated blood in a Vacutainer blood collection tube (Becton Dickinson, green top, lithium heparin as anticoagulant, each tube holds 7-mL). Spin down cell pellet at 1,000 rpm. Cells are resuspended in 5 mL PBS containing 0.5 mM EDTA and the mononucleate cell (MNC) fraction is obtained by Ficoll-Paque density centrifugation (Pharmacia) according to manufacturer's instruction, washed in complete culture medium containing 15% bovine serum, and suspended in 3-15 mL of the complete medium.

(2) Culture of the MNC fraction on a CAM 96-well chamber slide: Seed 100 μl/well of the cell suspension (also applicable to the cells captured by other methods such as CAM and Dynal AAMB) onto the remaining 88 wells of a 96-well microtiter plate that were coated with type I-collagen-based CAM that have been filled with 100 μl of complete culture medium containing 15% bovine serum and cultured in a CO₂ incubator at 37° C. for 12-18 hours.

(3) Non-adherent cells and supernatants are removed carefully by pipetting. Wash the wells 3 times in 200 μl of PBS without disturbing the CAM film on the inner wall. Non-adherent cells consist of dead tumor cells and non-tumor blood cells in the MNC fraction. Suspended cells can be pooled and subjected to cell isolation for CD 19 leukocytes or stem cells.

(4) Isolation of RNA for CAM-captured cells: Add 10 μL/well of Trizol reagent into each well of the 88-well row of the 96-well CAM blood unit that has been thoroughly washed. Total RNA is extracted using Trizol reagent (Invitrogen, Carlsbad, Calif.), followed by clean up on a RNeasy spin column (Qiagen, Inc., Valencia, Calif.).

### EXAMPLE 11

### Immunocytochemistry Using Cell Type Antibody Markers to Validate Purity of Cell Fractions

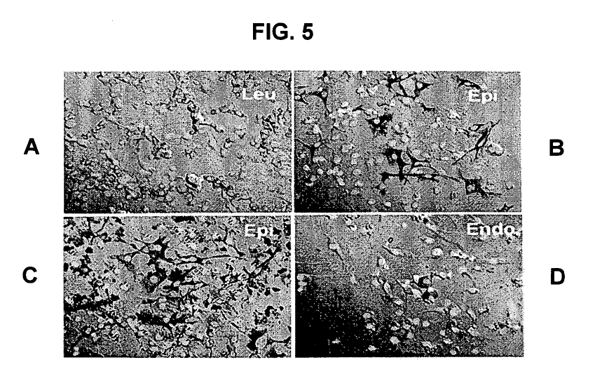

Immunocytochemistry using cell type antibody markers was used to validate the purity of cell fractions. The upper two panels of FIG. 5 show immuno-cytochemical identification of leukocytes (Leu) and tumor cells (Epi) enriched by CAM from ascites of serous adenocarcinoma of the ovary, using antibodies directed against CD45, a pan-leukocyte antigen (left panel, Leu, red), and antibodies against pan-cytokeratins, epithelial antigens (right panel, Epi, red). The lower two panels show immunocytochemical identification of pure tumor cells enriched by CAM and followed by antibody EPCAM positive-selection. Tumor cells labeled with antibodies against pan-cytokeratins now predominate (left panel, Epi, red). Note that some EpCAM antibody-Dynal beads are visible on tumor cells. A few (2%) of the pure tumor cells were labeled with antibodies directed against CD31 (right panel, Endo, red), an endothelial surface antigen. Nuclei were stained blue as a universal cell marker using Hoechst 33342 nuclear staining after permeablizing the plasma membrane with non-ionic detergents. (Picture size, 331 μm×239 μm.)

### EXAMPLE 12

### Real-Time RT-PCR Analysis

Real-time RT-PRC analysis may be used to further elucidate the genetic basis for one or more cancers. RT-PCR analysis may also be used to validate microarray data.

Quantitative real-time RT-PCR was used to measure the expression of 10 genes selected from DNA microarray clusters that were specific for the seven cell populations representative of 63 cell samples purified (FIG. 5A). (A) Quantitative real-time RT-PCR analysis of five genes up-regulated among the different tumor cell types (MMP7, mucin 1, GA733-1, lipocalin 2 and cytokeratin 18); four gene up-regulated among leukocytes (CD45, autotaxin, CXCR4 and SDF-1); one gene up-regulated among fibroblasts (type I collagen) on all 63 cell samples. (B) Quantitative real-time RT-PCR analysis of the ten genes differentially regulated among the seven cell groups. Bar graphic plot is used to demonstrate the typical gene expression patterns of different cell groups as well as fluctuations of expression levels within and between cell groups. For each gene, relative expression is compared with the mean fold expression (normalized to β-actin) of numbers of cell samples in each group. Error bars, SE of the means.

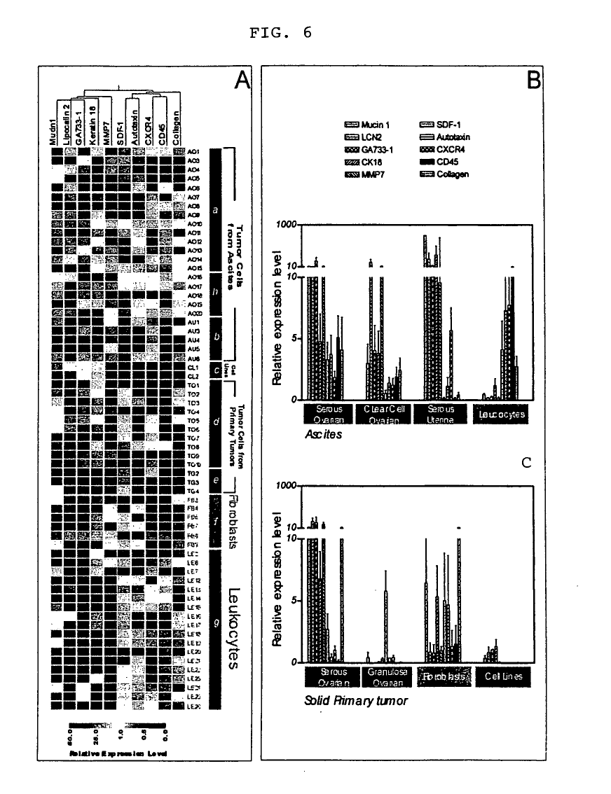

Of the four different types of tumor cells isolated by a CAM-initiated cell separation device, the five up-regulated genes were found to be highly expressed in most adenocarcinoma cell samples enriched from ovarian and uterine tumor specimens (FIG. 6A-6C). Expression differences beteen different types of cell groups for tumor cell-, leukocyte- and fibroblast-associated genes were also seen to be similar between DNA microarray data and real-time RT-PCR data. These results suggest that most array probe sets are likely to accurately measure the levels of the intended transcript within a complex mixture of transcripts.

It will be appreciated that various of the above-disclosed and other features and functions or alternatives thereof may be desirably combined into many other different systems or applications. Also, it will be appreciated that various presently unforeseen or unanticipated alternatives, modifications, variations or improvements therein may be subsequently made by those skilled in the art which are also intended to be encompassed by the following claims.

### REFERENCES

Aoyaina, A. and Chen, W.-T. (1990). A 170-kDa membrane-bound protease is associated with the expression of invasiveness by human malignant melanoma cells. Proc. Nati. Acad. Sci. U. S. A. 87, 8296-8300.
Asahara, T., Murohara, T., Sullivan, A., Silver, M., Van der Zee, R, Li, T., Witzenbichler, B., Schatteman, G., and Isner, J. M. (1997). Isolation of putative progenitor endothelial cells for angiogenesis. Science 275, 964-967.
Beitsch, P. D. and Clifford, E. (2000). Detection of carcinoma cells in the blood of breast cancer patients. American Journal of Surgery 180, 446-448.
Brandt, B. H., Scbmidt, H., de Angelis, G., and Zanker, K. S. (2001). Predictive laboratory diagnostics in oncology utilizing blood-borne cancer cells--current best practice and unmet needs. Cancer Letters 162 Supp, S11-S16.
Brugger, W., Bubring, H. J., Grnnebach, F., Vogel, W., Kaul, S., Muller, R, Brummendorf, T. H., Ziegler, B. L., Rappold, I., Brossart, P., Scheding, S., and Kutnz, L. (1999). Expression of MUC-I epitopes on normal bone marrow: implications for the detection of micrometastatic tumor cells. J. Chin. Oncol. 17, 1535-1544.
Chadha M, Chabon A B, Friedmann P, and Vikram B (1994). Predictors of axillary lymph node metastases in patients with TI breast cancer. A multivariate analysis. Cancer 73, 350-353.
Chen, J. M. and Chen, W. T. (1987). Fibronectin-degrading proteases from the membranes of transformed cells. Cell. 48, 193-203.
Chen, W. T. (1989). Proteolytic activity of specialized surface protrusions formed at rosette contact sites of transformed cells. J. Exp. Zool. 251, 167-185.
Chen, W. T. (1996). Proteases associated with invadopodia, and their role in degradation of extracellular matrix. Enzyme Protein 49, 59-71.
Chen, W. T., Lee, C. C., (ioldstein, L., Bemier, S., Liu, C. H., Lin, C. Y., Yeh, Y., Monsky, W. L., Kelly, T., Dai, M., and Mueller, S. C. (1994b). Membrane proteases as potential diagnostic and therapeutic targets for breast malignancy. Breast Cancer Res. Treat. 31, 217-226.
Chen, W. T., Olden, K˜, Bernard, B. A., and Chu, F. F. (1984). Expression of transformation-associated protease(s) that degrade fibronectin at cell contact sites. J. Cell Biol 98, 1546-1555.
Chen, W. T. and Wang, J. Y. (1999). Specialized surface protrusions of invasive cells, invadopodia and lamelhipodia, have differential MTI-MMP, MMP-2, and TIMP-2 localization. [Review] [52 refs]. Annals of the New York Academy of Sciences 878, 361-371.
Chen, W. T., Yeh, Y., and Nakahara, H. (1994a). An in vitro cell invasion assay: determination of cell surface proteolytic activity that degrades extracellular matrix. J. Tiss. Cult. Meth. 16, 177-181.
Compton, C. C. (2003). Colorectal Carcinoma: Diagnostic, Prognostic, and Molecular Features. Mod Pathol 16, 376.
Feezor, R. J., Copeland, E. M., III, and Hochwald, S. N. (2002). Significance of Micrometastases in Colorectal Cancer. Ann Surg Oncol 9, 944-953.
Fehm, T., Sagalowsky, A., Clifford, E., Beitsch, P., Saboorian, H., Euhus, D., Meng, S., Morrison, L., Tucker, T., Lane, N., Ghadimi, B. M., Hesehneyer-Haddad, K., Ried, T., Rao, C., and Ubr, J. (2002). Cytogenetic Evidence That Circulating Epithelial Cells in Patients with Carcinoma Are Malignant. Clinical Cancer Research 8, 2073.
Flatmnark, K., Bjornland, K., Johannessen, H. O., Hegstad, E., Rosales, R, Harklau, L., Solhaug, J. H., Faye, R S., Soreide, O., and Fodstad, O. (2002). Iinmunomagnetic Detection of Micrometastatic Cells in Bone Marrow of Colorectal Cancer Patients. Clinical Cancer Research 8,444-449.
Ghersi, G., Dong, H., Goldstein, L. A., Yeh, Y., Hakkinen, L., Larjava, H. S., and Chen, W. T. (2002). Regulation of fibroblast migration on collagenous matrix by a cell surface peptidase complex. J. Biol. Chem. 277, 29231-29241.
Ahossein, R A., Bhattacharya, S., and Rosai, J. (1999). Molecular detection of micrometastases and circulating tumor cells in solid tumors. Chin Cancer Res 5, 1950-1960.
Glaves, D. (1983). Correlation between circulating cancer cells and incidence of metastases. British Journal of Cancer 48, 665-673.
Glaves, D., Huben, R P., and Weiss, L. (1988). Haematogenous dissemination of cells from human renal adenocarcinomas. British Journal of Cancer 57, 32-35.
Goldstein, L. A. and Chen, W. T. (2000). Identification of an alternatively spliced seprase mRNA that encodes a novel intracellular isoform. J. Biol. Chem. 275, 2554-2559.
Goldstein, L. A. and Chen, W. T. (1997). Molecular cloning of seprase: A seine integral membrane protease from human melanoma. Biochimica et Biophysica Acta 1361, 11-19.
Gross, H., Verwer, B., Houck, D., Hoffinan, R A., and Recktenwald, D. (1995). Model Study Detecting Breast Cancer Cells in Peripheral Blood Mononuclear Cells at Frequencies as Low as 10-7. PNAS 92, 537-541.
Gulati, S. C. (1993). Questioning the role of purging in BMT. [Review] [11 refs]. Stem Cells 11, 249-251.
Gulati, S. C. and Acaba, L. (1993). Rationale for purging in autologous stem cell transplantation. [Review] [17 refs]. Journal of Hematotherapy 2, 467-471.
Hill, J. M., Zalos, G., Halcox, J. P. J., Schenke, W. H., Waclawiw, M. A., Quyyumi, A. A., and Finkel, T. (2003). Circulating Endothehial Progenitor Cells, Vascular Function, and Cardiovascular Risk. N Engl J Med 348, 593-600.
Karczewski, D. M., Lema, M. J., and Giaves, D. (1994). The efficiency of an autotransfusion system for tumor cell removal from blood salvaged during cancer surgery. Anesthesia & Analgesia 78, 1131-1135.
Kelly, T., Mueller, S. C., Yeh, Y., and Chen, W. T. (1994). Invadopodia promote proteolysis of a wide variety of extracellular matrix proteins. J. Cell Physiol. 158, 299-308.
Koch, M., Weitz, J., Kienle, P., Benner, X, Willeke, F., Lebnert, T., Herfarth, C., and Knebel Doeberitz, M. (2001). Comparative Analysis of Tumor Cell Dissemination in Mesenteric, Central, and Peripheral Venous Blood in Patients With Colorectal Cancer. Arch Suing 136, 85.
Liefers, G. J., Cleton-Jansen, A. M., van de Velde, C. J., Hermans, J., van Krieken, J. H., Comeisse, C. J., and Tollenaar, R A. (1998). Micrometastases and survival in stage II colorectal cancer. N. Engl. J Med. 339, 223-228.
Luzzi, K. J., MacDonald, I. C., Schinidt, E. E., Kerkvliet, N., Morris, V L, Chambers, A. F., and Groom, A. C. (1998). Multistep nature of metastatic inefficiency: dormancy of solitary cells after successful extravasation and limited survival of early micrometastases. Am J Pathol 153, 865-873.
Matsunami, K, Nakamura, T., Oguma, H., Kitamura, Y., and Takasaki, K. (2003). Detection of Bone Marrow Micrometastasis in Gastric Cancer Patients by Immunomagnetic Separation. Ann Suing Oncol 10, 171.
Molnar, B., Ladanyi, A., Tanko, L., Sreter, L., and Tulassay, Z. (2001). Circulating Tumor Cell Clusters in the Peripheral Blood of Colorectal Cancer Patients. Clinical Cancer Research 7, 4080.
Monsky, W. L., Kelly, T., Lin, C. Y., Yeh, Y., Stetler-Stevenson, W. G., Mueller, S. C., and Chen, W.-T. (1993). Binding and localization of M(r) 72,000 matrix metalloproteinase at cell surface invadopodia. Cancer Res. 53, 3 159-3164.
Monsky, W. L., Lin, C.-Y., Aoyama, N, Kelly, T., Mueller, S. C., Akiyama, S. K, and Chen, W.-T. (1994). A potential marker protease of invasiveness, seprase, is localized on invadopodia of human malignant melanoma cells. Cancer Res. 54, 5702-5710.
Mueller, S. C. and Chen, W. T. (1991). Cellular invasion into matrix beads: localization of beta 1 integrins and fibronectin to the invadopodia. J. Cell Sci 99, 2 13-225.
Mueller, S. C., Ghersi, G., Akiyama, S. K., Sang, Q. X., Howard, L., Pineiro-Sanchez, M., Nalcahara, H., Yeh, Y., and Chen, W.-T. (1999). A novel protease-docking function of integrin at invadopodia. J. Biol. Chem. 274, 24947-24952.
Mueller, S. C., Yeh, Y., and Chen, W.-T. (1992). Tyrosine phosphorylation of membrane proteins mediates cellular invasion by transformed cells. J. Cell Biol 119, 1309-1325.
Nakahara, H., Howard, L., Thompson, E. W., Sato, H., Seiki, M., Yeh, Y., and Chen, W. T. (1997). Transniembrane/cytoplasmic domain-mediated membrane type 1-matrix metalloprotease docking to invadopodia is required for cell invasion. Proc. Natl. Acad. Sci. U. S. A. 94, 7959-7964.
Nakahara, H., Mueller, S. C., Nomizu, M., Yamada, Y., Yeh, Y., and Chen, W. T. (1998). Activation ofbetal integrin signaling stimulates tyrosine phosphorylation ofpI9ORhoGAP and membrane-protrusive activities at invadopodia. Journal of Biological Chemistry 273, 9-12.
Nakahara, H., Nomizu, M., Akiyama, S. K, Yamada, Y., Yeh, Y., and Chen, W.-T. (1996). A mechanism for regulation of melanoma invasion. Ligation of alpha6betal integrin by laminin G peptides. J. Biol. Chem. 271, 27221-27224.
Nomoto, S., Nakao, A., Ando, N., Takeda, S., Kasai, Y., Inoue, S., Kaneko, T., and Takagi, H. (1998). Clinical application of K-ras oncogene mutations in pancreatic carcinoma: detection of micrometastases. Seminars in Surgical Oncology 15, 40-46.
Olivier, et al. (1999). A rapid single-laser flow cytometric method for discrimination of early apoptotic cells in a heterogenous cell population. Br J Haematol 104, 530-537.
Pantel, K., Cote, R J., and Fodstad, O. (1999). Detection and clinical importance of micrometastatic disease. J. Natl. Cancer Inst. 91, 1113-1124.
Pavlaki, M., Cao, J., Hymowitz, M., Chen, W. T., Bahou, W., and Zucker, S. (2002). A Conserved Sequence within the Propeptide Domain of Membrane Type 1 Matrix Metalloproteinase Is Critical for Function as an Intramolecular Chaperone. Journal of Biological Chemistry 277, 2740-2749.
Peck, K., Sher, Y. P., Shih, J. Y., Roffier, S. R, Wu, C. W., and Yang, P. C. (1998). Detection and quantitation of circulating cancer cells in the peripheral blood of lung cancer patients. Cancer Res. 58, 2761-2765.
Pineiro-Sanchez, M. L., Goldstein, L. A., Dodt, J., Howard, L., Yeh, Y., Tran, H., Argraves, W. S., and Chen, W. T. (1997). Identification of the 170-kDa melanoma membrane-bound gelatinase (seprase) as a seine integral membrane protease. J. Biol. Chem. 272, 7595-7601; Correction (1998) J. Biol. Chem. 273, 13366.
Racila, E., Euhus, D., Weiss, A. J., Rao, C., McConnell, J., Terstappen, L. W., and Uhr, J. W. (1998). Detection and characterization of carcinoma cells in the blood. Proceedings of the National Academy of Sciences of the United States of America 95, 45894594.
Rill, D. R., Santana, V. M., Roberts, W. M., Nilson, T., Bowman, L. C., Krance, R. A., Heslop, H. E., Moen, R C., Jihle, J. N., and Brenner, M. K. (1994). Direct demonstration that autologous bone marrow transplantation for solid tumors can return a multiplicity of tumorigenic cells. Blood 84, 380-383.
Ross, R. (1993). The pathogenesis of atherosclerosis: a perspective for the 1990s. Nature 362, 801-809.
Sabile, A., Louha, M., Bonte, E., Poussin, K., Vona, G., Mejean, A., Chretien, Y., Bougas, L., Lacour, B., Capron, F., Roseto, A., Brechot, C., and Paterlini-Binechot, P. (1999). Efficiency of Ber-EP4 antibody for isolating circulating epithelial tumor cells before RT-PCR detection. American Journal of Clinical Pathology 112, 171-178.
Saga, S., Chen, W. T., and Yamada, K. M. (1988). Enhanced fibronectin receptor expression in Rous sarcoma virus- induced tumors. Cancer. Res. 48, 5510-5513.
Schoenfeld, A., Kruger, I C H., Gomm, J., Sinnett, H. D., Gazet, J. C., Sacks, N., Bender, H G, Luqmani, Y., and Coombes, R C. (1997). The detection of micrometastases in the peripheral blood and bone marrow of patients with breast cancer using immunobistochemistry and reverse transcriptase polymerase chain reaction for keratin 19. European Journal of Cancer 33, 854-861.
Soeth, E., Vogel, I., Roder, C., Julil, H., Marxsen, J., Kruger, U., Henne-Bruns, D., Kremer, B., and Kalthoff, H. (1997). Comparative analysis of bone marrow and venous blood isolates from gastrointestinal cancer patients for the detection of disseminated tumor cells using reverse transcription PCR. Cancer Research 57, 3106.
Suarez-Quian, C. A., Goldstein, S. R., Pohida, T., Smith, P. D., Peterson, J. I., Welluer, E, Ghany, M., and Bonner, R F. (1999). Laser capture microdissection of single cells from complex tissues. BioTechniques 26, 328-335.
Szmitko, P. E., Fedak, P. W. M., Weisel, R D., Stewart, D. J., Kutryk, M. J. B., and Verma, S. (2003). Endothelial Progenitor Cells: New Hope for a Broken Heart. Circulation 107, 3093-3100.
Vlems, F. A., Diepstra, J. H. S., Cornelissen, I. M. H. A., Ruers, T. J. M., Ligtenberg, M. J. L., Punt, C. J. A., Van Krieken, J. H. J. M., Wobbes, T., and Van Muijen, G. N. P. (2002). Limitations of cytokeratin 20 RT-PCR to detect disseminated tumour cells in blood and bone marrow of patients with colorectal cancer: expression in controls and dowuregulation in tumour tissue. Mol Pathol 55, 156.
Vona, G., Sabile, X, Louha, M., Sitruk, V., Romana, S., Schutze, K., Capron, F., Franco, D., Pazzagli, M., Vekemans, M., Lacour, B., Brechot, C., and Paterlini-Brechot, P. (2000). Isolation by Size of Epithelial Tumor Cells: A New Method for the Immunomorphological and Molecular Characterization of Circulating Tumor Cells. Am J Pathol 156, 57-63.
Walsh, J. M. E. and Terdiman, J. P. (2003). Colorectal Cancer Screening: Clinical Applications. JAMA: The Journal of the American Medical Association 289, 1297.
Wang, Z. P., Eisenberger, M A, Carducci, M A, Partin, A. W., Scher, Hi, and Ts'o,P.O. (2000). Identification and characterization of circulating prostate carcinoma cells. Cancer 88, 2787-2795.
Weihrauch, M. R (2002). Immunomagnetic Enrichment and Detection of Micrometastases in Colorectal Cancer: Correlation With Established Clinical Parameters. [Report]. J. Chin. Oncol. 20, 4338-4343.
Weitz, J., Kienle, P., Magener, A., Koch, M., Schrodel, A., Willeke, F., Autschbach, F., Lacroix, J., Lehnert, T., Herfarth, C., and Doeberitz, M. v. K. (1999). Detection of Disseminated Colorectal Cancer Cells in Lymph Nodes, Blood and Bone Marrow. Clinical Cancer Research 5, 1830.
Wharton, R. Q., Jonas, S. K., Glover, C., Khan, Z. A. J., Klokouzas, A., Quinn, H., Henry, M., and AllenMersh, T. G. (1999). Increased Detection of Circulating Tumor Cells in the Blood of Colorectal Carcinoma Patients Using Two Reverse Transcription-PCR Assays and Multiple Blood Samples. Clinical Cancer Research 5, 4158.
Wilhelm M C, Edge S B, Cole D D, deParedes E, and Frierson H F Jr (1991). Nonpalpable invasive breast cancer. Am Surg. 213, 600-603.
Zucker, S., Cao, J., and Chen, W. T. (2000). Critical appraisal of the use of matrix metalloproteinase inhibitors in cancer treatment. Oncogene 19, 6642-6650.
Zukowska-Grojec, Z., Karwatowska, P., Rose, W., Rone, J., Movafagh, S., Ji, H., Yeh, Y., Chen, W.-T., Kleinman, H. K., Grouzrnann, E., and Grant, D. S. (1998). Neuropeptide Y: a novel angiogenic factor from the sympathetic nerves and endothelium. Circ. Res. 83, 187-195.

1. An apparatus for isolating target cells from a fluid sample, comprising:
a vessel, having an inner surface and an outer surface;
a cell adhesion matrix comprising a non-reactive core material associated with one or more cell adhesion molecules;
wherein said cell adhesion matrix is coated on said inner surface of said vessel.

2. An apparatus of claim 1 wherein the non-reactive core material of said cell adhesion matrix, is at least one of a material selected from the group consisting of: gelatin, cross-linked gelatin, bone, glass, inert polymers, and dextran.

3. An apparatus of claim 1 wherein said cell adhesion molecule of said cell adhesion matrix is at least one molecule selected from the group consisting of: proteoglycan, fibronectin, fibrin, heparin, lamimin, tenascin, vitronectin, and/or fragments thereof.

4. An apparatus of claim 1 wherein the inner surface of said vessel is at least 5% coated with said cell adhesion matrix.

5. The apparatus of claim 1 further comprising at least one ligand having affinity for said target cell(s) that is detectible when associated with said target cell(s).

6. The apparatus of claim 5 wherein said ligand is fluorescently-labeled.

7. The apparatus of claim 5 wherein said ligand is integrated into said cell adhesion matrix.

8. The apparatus of claim 5 wherein said ligand is found in a layer associated with said cell adhesion matrix.

9. An apparatus of claim 1 further comprising a cell separation mechanism proximal to said cell adhesion matrix, said cell separation mechanism operatively configured to remove cells from a sample containing said target cells prior to interaction of said target cells with said cell adhesion matrix.

10. The apparatus of claim 9 wherein said cell separation mechanism is at least one mechanism selected from the group consisting of: a filter, a membrane, a mesh, a material gradient.

11. The apparatus of claim 5 wherein at least one ligand is operatively configured to permit visual detection upon the interaction of the ligand with an isolated target cell.

12. A method employing the apparatus of claim 1 comprising:
contacting a mixture of cells in said fluid sample to said cell adhesion matrix in said apparatus;
isolating target cells from said cell adhesion matrix.

13. The method of claim 12, further comprising removing unbound cells from said cell adhesion matrix.

14. The method of claim 12, wherein said fluid sample is a blood sample or an ascites sample or biopsy or scrape or smear sample.

15. The method of claim 12, wherein said cell mixture comprises mononucleated cells from a blood sample after density gradient centrifugation or red cell lysis.

16. The method of claim 12, wherein said target cells are tumor cells, endothelial cells or fetal cells.

17. The method of claim 16, wherein the tumor cells are derived from cancer of at least one of the lungs, bladder, mammary tissue, ovary, prostate, pancreas, breast, skin, liver, stomach, esophagus, head-and-neck, cervix, uterus, brain, kidney, thyroid, colon or rectum.

18. The method of claim 12, wherein the target cells are endothelial cells or endothelial progenitor cells.

19. The method of claim 12, wherein the target cells are fetal cells obtained from a pregnant female.

20. The method of claim 12, wherein said cell adhesion matrix comprises beads.

21. The method of claim 12, wherein said cell adhesion matrix comprises a fluorescently labeled cell adhesion matrix component.

22. The method of claim 12, wherein said target cells comprise invadopodia.

23. The method of claim 12, wherein the target cells comprise cell adhesion receptor integrins.

24. A vessel having an opening, a bottom, and surrounding side walls, and comprising at least one coating layer of a cell adhesion matrix on the inner surface of said vessel which is operatively configured to be contacted by a fluid sample when fluid is placed into the opening of said vessel.

25. The vessel of claim 24, wherein said vessel is selected from the group consisting of: a microtiter plate, a microscope slide chamber, a tissue culture device, a cell chamber unit, a blood filtration unit, a tube, bottle, or combinations thereof.

26. The fluorescently labeled cell adhesion matrix of claim 21, wherein said matrix is used to label a cancer cell in blood.

27. A method for prenatal diagnosis of disease, comprising:
contacting a blood sample from a pregnant female with a cell adhesion matrix,
isolating said fetal cells from said cell adhesion matrix,
culturing said fetal cells in a medium, and
testing said fetal cells for the presence of genetic and chromosomal abnormalities.

28. The method of claim 27, wherein the genetic and chromosomal abnormalities are selected from the group consisting of: Down's Syndrome, Marfan's syndrome, Taysach's disease, and thalasemias.

29. The method of claim 27, wherein said cell adhesion matrix comprises a plurality of coated beads comprising a non-reactive core material and cell adhesion molecules surrounding said core material.

30. The cell adhesion matrix of claim 29, wherein said non-reactive core is at least one material selected from the group consisting of: collagen microbeads, gelatin microbeads and glass microbeads, or combinations thereof.

31. The vessel of claim 30, wherein the collagen is labeled with a fluorescent dye.

32. A method for diagnosing cancer in vitro, comprising:
contacting a sample fluid obtained from a patient with a cell adhesion matrix comprising blood-borne components;
isolating metastatic tumor cells adhered to said matrix from cells in said sample fluid;
culturing said metastatic tumor cells adhered to said matrix for a predetermined period of time; and
performing microscopic and flow cytometric analyses of said metastatic tumor cells in said culture.

33. The method of claim 32, wherein said method further comprises the step of performing immunocytochemistry on the metastatic cancer cells and/or staining said cancer cells with labeled cell adhesion matrix and nucleic acid dyes to identify the type of cancer cell present in said sample fluid.

34. The method of claim 32, wherein said method further comprises characterizing said metastatic tumor cells using DNA microarray analysis and/or real-time PCR quantification of an epithelial tumor gene marker.

35. The method of claim 34, wherein the tumor gene markers are GA733-2, GA733-1, MMP7, mucin 1, lipocalin 2 an cytokeratin 18, E-cadherin-1, seprase, autotoxin and CXCR4.

36. A filtration cassette housing a fluid inlet and a fluid outlet, said housing comprising:
a pre-filter proximal to said fluit inlet;
a post-filter proximal to said fluid outlet; and
a filter compartment comprising a cell adhesion matrix, said filter compartment being positioned between said pre-filter and said post-filter.

37. The method of claim 36 wherein one of said pre-filter or said post-filter is associated with a cell adhesion matrix.

38. An apparatus for isolating target cells from a fluid sample, comprising:
a vessel having an inner surface designed to hold said fluid sample and an outer surface;
a dipstick comprising a lid connected to a card having a cell adhesion matrix.
